SEQUENCE LISTING (6 sequences)

SEQ ID NO: 1 - DNA, 40 bp, Artificial Sequence
  Synthetic Polynucleotide
  MISC_Feature (20)..(20): T(DBCO)-CY3
aacatcttgtgctcaatatctagtcgtctacgtaacagtc

SEQ ID NO: 2 - DNA, 40 bp, Artificial Sequence
  Synthetic Polynucleotide
  MISC_Feature (20)..(20): T(DCBO)-CY5
ttaggctggatctcgcgttctgcacagacccatgtactcg

SEQ ID NO: 3 - DNA, 40 bp, Artificial Sequence
  Synthetic Polynucleotide
  MISC_Feature (20)..(20): T(DBCO)-FITC
taactcgtgaacgtatgctcaatcatcagtactcacctag

SEQ ID NO: 4 - DNA, 48 bp, Artificial Sequence
  Synthetic Polynucleotide
gtaacgatgatattgagcacaagatgttctaggtgagtactgatgatt

SEQ ID NO: 5 - DNA, 48 bp, Artificial Sequence
  Synthetic Polynucleotide
cattcagagaacgcgagatccagcctaagactgttacgtagacgacta

SEQ ID NO: 6 - DNA, 48 bp, Artificial Sequence
  Synthetic Polynucleotide
gtcatgttgagcatacgttcacgagttacgagtacatgggtctgtgca

(19) United States
(12) Patent Application Publication
(10) Pub. No.: US 2024/0148877 A1
(43) Pub. Date: May 9, 2024

(54) Synthetic Strategy to Polymerize Protein into Molecularly Defined Polymers

(71) Applicant: NORTHWESTERN UNIVERSITY, Evanston, IL (US)

(72) Inventors: Chad A. Mirkin, Wilmette, IL (US); Peter H. Winegar, Evanston, IL (US); Charles A. Figg, Evanston, IL (US)

(21) Appl. No.: 18/283,241

(22) PCT Filed: Mar. 22, 2022
(86) PCT No.: PCT/US22/21359

§ 371 Date: Sep. 21, 2023

Related U.S. Application Data
(60) Provisional application No. 63/247,549, filed on Sep. 23, 2021.
(60) Provisional application No. 63/164,214, filed on Mar. 22, 2021.

(51) Int. Cl. A61K 47/54; C07K 16/46

(52) U.S. Cl. CPC A61K 47/549; C07K 16/46

(57) ABSTRACT

Methods for forming a sequence encoded oligomer includes oligomerizing first and second antibody-oligonucleotide conjugates through hybridization using at least one template oligo nucleotide. The first antibody-oligonucleotide conjugate comprises an oligonucleotide strand comprising a first sequence and a second sequence joined by an alkyne. The second antibody-oligonucleotide conjugate comprises an oligonucleotide strand comprising a third sequence and a fourth sequence joined by an alkyne. The at least one template oligonucleotide strand comprises at least two template sequences, one of the t two template sequences being complementary to one of the first and second sequences of the first antibody-oligonucleotide conjugate and the other being complementary to one of the third and fourth sequences of the second antibody-oligonucleotide conjugate, such that the at least one template oligonucleotide strand hybridizes to a portion of the oligonucleotide strand of the first-antibody-oligonucleotide conjugate and to a portion of the oligonucleotide strand of the second-antibody.

### CROSS-REFERENCE TO RELATED APPLICATIONS

The benefit of priority to U.S. Provisional Patent Application No. 63/164,214 filed Mar. 22, 2021 and U.S. Provisional Patent Application No. 63/247,549 filed Sep. 23, 2021 is hereby claimed and the respective disclosures are each incorporated herein by references in their entireties.

### STATEMENT OF GOVERNMENT SUPPORT

This invention was made with government support under N00014-16-1-3117 awarded by The Office of Naval Research (ONR). The government has certain rights in the invention.

### INCORPORATION BY REFERENCE OF THE SEQUENCE LISTING

This application contains, as a separate part of disclosure, a Sequence Listing in computer-readable form (filename: 2021-067_Seqlisting.txt; Size: 1,718 bytes: Created: Mar. 22, 2022) which is incorporated by reference herein in its entirety.

### BACKGROUND

Antibodies are proteins from the immune system that bind to targets with high specificity and affinity and can elicit specific responses from the innate and adaptive immune systems. Monoclonal antibody therapeutics that either repurpose known antibodies or are engineered to bind novel targets have been approved and are in clinical trials to treat a range of diseases. Co-administering two separate antibodies can enhance therapeutic activity because multiple biological redundancies can be targeted. For example, the co-administration of two antibodies that inhibit distinct checkpoint pathways is approved to treat advanced melanoma and is in clinical trials to treat locally advanced or metastatic non-small cell lung cancers. Furthermore, tethering antibodies into oligomers prior to co-administration can enhance therapeutic efficacy through cooperative binding to targets. Bivalent and trivalent antibodies have been engineered to contain distinct recognition sites within a single construct and these dual- or tri-affinity constructs display emergent properties, such as dual antigen recognition or cell tethering capabilities. However, each oligomeric, bivalent, or trivalent antibody construct must be rigorously engineered using techniques from molecular biology. Bivalent antibodies can be readily synthesized using biorthogonal conjugation reactions to dimerize antibodies. For example, two antibodies can be conjugated using a linker molecule containing bis-maleimide moieties. However, larger oligomers cannot be synthesized using these approaches.

The sequence specificity of DNA has been used in combination with biorthogonal chemistry to program the nanoscale organization of nanomaterials, including antibodies. For example, DNA-DNA interactions have been used to synthesize antibody tetrahedra, antibody hetero-dimers, and sensing architectures that contain antibodies. However, precisely defining the specific number and sequence of antibodies within antibody oligomers has not been possible with these approaches because they use nonmodular DNA scaffolds where the scaffold architecture defines the exact number of possible antibodies. A DNA tetrahedra can only be functionalized with four antibodies. DNA-DNA interactions have also been used to organize other proteins into specific nanoscale architectures.

Previous work has used recombinant protein expression to prepare mutated proteins that contain specific chemical sites for functionalization with DNA. Subsequently, DNA-DNA interactions are used to organize protein-DNA conjugates into protein polymers, where there is a statistical number distribution of proteins per polymer. Others have functionalized proteins with DNA and used DNA-DNA interactions to bind protein-DNA conjugates to DNA nanostructure scaffolds. Others have used rolling-circle amplification of a circular DNA template to prepare a DNA scaffold that contains periodically arranged protein-binding DNA aptamers. Protein polymers are prepared through protein-aptamer binding between proteins and the DNA scaffold.

Certain protein pairs react to form irreversible isopeptide bonds. Others have used recombinant protein expression to prepare protein fusions between such proteins and proteins of interest. Isopeptide bond formation between fusion proteins leads to the formation of protein oligomers.

Certain enzyme-substrate pairs react to form irreversible covalent bonds. Others have used recombinant protein expression to prepare protein fusions between such enzymes and proteins of interest. Covalent bond formation between fusion proteins and substrates leads to the formation of protein oligomers.

### SUMMARY

Disclosed herein are generalizable and modular methods to synthesize antibody oligomers with defined sequences and numbers of distinct antibodies, antibodies which could be therapeutically relevant. Methods of the disclosure can utilize modular DNA scaffolds to organize antibodies into sequence-encoded oligomers, where both the number and sequence of antibodies can be controlled using DNA-DNA interactions.

This disclosure provides a synthetic strategy to polymerize nearly any protein or set of proteins into molecularly defined polymers containing specific sequences, stoichiometries, and architectures. For example, first, proteins can be conjugated with synthetic oligonucleotides to prepare protein-DNA conjugates. Next, standard protein purification techniques are utilized to isolate protein-DNA conjugates that consist of one DNA strand per protein. Finally, DNA-DNA interactions are used to organize protein-DNA conjugates into sequence-encoded protein polymers. Taken together, this technology provides a modular technique to synthesize protein oligomers with nearly any known protein and could have implications for catalysis, materials, and therapeutics.

### BRIEF DESCRIPTION OF THE DRAWINGS

FIG. 1A is a schematic illustration of functionalization of antibodies with a single DNA strand. Primary amines on the surface of the antibodies were functionalized with azides using an NHS-PEG₁₂-azide linker. Dibenzocyclooctyne (DBCO)-modified DNA was conjugated to azide-modified antibodies via a strain promoted azide-alkyne cycloaddition (SPAAC).

FIG. 1B shows an SDS PAGE characterization of mouse antibodies (i.e., anti-mouse-PD-1 in lane 1, anti-mouse-TIGIT in lane 3, and anti-mouse-CTLA-4 in lane 5) and mouse antibody-DNA conjugates (i.e., S2-anti-mouse-PD-1-Cy3-S3 in lane 2, S4-anti-mouse-TIGIT-Cy5-S5 in lane 4, S6-anti-mouse-CTLA-4-FITC-S1 in lane 6) functionalized in accordance with the schematic of FIG. 1A. A single gel was imaged for SimplyBlue SafeStain, Cy3, Cy5, and FITC fluorescence.

FIG. 2A is a schematic illustration of mouse antibody DNA conjugates and template DNA strands using in assembly of sequence encoded oligomers.

FIG. 2B is schematic illustration of a method of forming a dimer using the conjugates and template DNA strands of FIG. 2A in accordance with the disclosure.

FIG. 2C is a schematic illustration of a method of forming a trimer using the conjugates and template DNA strands of FIG. 2A in accordance with the disclosure.

FIG. 2D is schematic illustration of a method of forming a pentamer using the dimers and trimers of FIGS. 2B and 2C, respectively, in accordance with the disclosure.

FIG. 2E is an image of agarose gel characterization of mouse antibody-DNA conjugates (i.e., S2-anti-mouse-PD-1-Cy3-S3 in lane 1, S4-anti-mouse-TIGIT-Cy5-S5 in lane 2, S6-anti-mouse-CTLA-4-FITC-S1 in lane 3), a mouse antibody dimer (i.e., S4-anti-mouse-TIGIT-anti-mouse-CTLA-4-S2′ in lane 4), a mouse antibody trimer (i.e., S2-anti-mouse-PD-1-anti-mouse-TIGIT-anti-mouse-CTLA-4-S1 in lane 5), and a mouse antibody pentamer (i.e., S4-anti-mouse-TIGIT-anti-mouse-CTLA-4-anti-mouse-PD-1-anti-mouse-TIGIT-anti-mouse-CTLA-4-S1 in lane 6).

FIG. 3A are schematic illustrations of human antibody-DNA conjugates tested for binding performance.

FIG. 3B is an agarose gel characterization of the human antibody-DNA conjugates of FIG. 3A, showing the antibodies retain binding after oligomerization (i.e., S2-anti-human-PD-1-Cy3-S3 in lane 1, S4-anti-human-PD-1-Cy5-S5 in lane 2, S6-anti-human-PD-1-FITC-S1 in lane 3), a human antibody dimer (i.e., S2-anti-human-PD-1-anti-human-PD-1-S5 in lane 4), and a human antibody trimer (i.e., S2-anti-human-PD-1-anti-human-PD-1-anti-human-PD-1-S1 in lane 5). A single gel was imaged for Cy3, Cy5, and FITC fluorescence and these images are merged into one composite image in FIG. 12.

FIG. 3C includes representative confocal microscopy images of untreated human peripheral blood mononuclear cells (hPMBCs) with upregulated PD-1 and the same cells treated with a human anti-human-PD-1-anti-human-PD-1-anti-human-PD-1 trimer at an overall antibody concentration of 50 nM. Cells were imaged for Cy3, Cy5, FITC, and CD8 stain and these images are merged into one composite image. Images for each sample were taken using the same laser settings and were processed identically.

FIG. 4A-4C are illustrations of a synthesis scheme for sequence-encoded antibody oligomers of the disclosure.

FIG. 5A is a graph showing normalized absorption as a function of time from an analytical SEC characterization of mouse antibody-DNA conjugations for the samples schematically illustrated in FIG. 5C.

FIG. 5B is a graph showing normalized fluorescence as a function of time from the SEC analytical characterization for a subset of the samples of FIG. 5C.

FIG. 5C is a schematic illustration of mouse-antibody DNA conjugates. for

FIG. 6 is an SDS-PAGE gel of human antibody functionalization with a single DNA strand. A single gel was imaged for SimplyBlue SafeStain, Cy3, Cy5, and FITC fluorescence.

FIG. 7A is a graph showing normalized absorbance as a function of time from an analytical SEC characterization of human antibody-DNA conjugation for the samples schematically illustrated in FIG. 7C.

FIG. 7B is a graph showing normalized fluorescence as a function of time from the analytical SEC characterization for a subset of the samples schematically illustrated in FIG. 7C.

FIG. 7C is a schematic illustration of human antibody-DNA conjugates.

FIG. 8A is a scheme showing oligomerization of mouse-antibody conjugates.

FIG. 8B is an agarose gel characterization of the mouse antibody-DNA conjugates and a mouse antibody trimer as illustrated in the scheme of FIG. 8A. A single gel was imaged for Cy3, Cy5, and FITC fluorescence.

FIG. 8C is analytical SEC characterization of the mouse antibody-DNA conjugates and the mouse antibody trimer of FIG. 8B.

FIG. 9A is a scheme showing oligomerization of human antibody-DNA conjugates.

FIG. 9B is an agarose gel characterization of the human antibody-DNA conjugates and a human antibody dimer as illustrated in the scheme of FIG. 9A.

FIG. 9C is analytical SEC characterization of the human antibody-DNA conjugates and the human antibody dimer of FIG. 9B.

FIG. 10 is a merged agarose gel image of mouse antibody-DNA oligomers of the Cy3, Cy5 and FITC fluorescence shown in FIG. 2E.

FIG. 11A is an illustration of assembly of human antibody-DNA conjugates into a human antibody dimer in accordance with methods of the disclosure.

FIG. 11B is an illustration of assembly of human antibody-DNA conjugates into a human antibody trimer in accordance with methods of the disclosure.

FIG. 12 is a merged agarose gel image of human antibody-DNA oligomers showing the fluorescence images of FIG. 3A.

FIG. 13 is a confocal microscopy image of untreated human peripheral blood mononuclear cells (hPMBCs) with upregulated PD-1 and hPMBCs treated with anti-human-PD-1, anti-human-PD-1-DNA conjugate, anti-human-PD-1 dimer, and anti-human-PD-1 trimer. Cells were imaged for Cy3, Cy5, FITC, and CD8. The Cy3, Cy5, FITC, and CD8 stain images were also merged into one composite image. Images for each sample were taken using the same laser settings and were processed identically.

FIG. 14 is a graph and associated data table showing the characterization of antibody function after oligomerization using PD-1 Blockade Bioassay.

FIG. 15A is an agarose gel characterization of oligomer degradation in 10% fetal bovine serum imaged for Cy5 fluorescence.

FIG. 15B is an agarose gel characterization of the samples of FIG. 15B imaged for FITC fluorescence.

FIG. 15C is a schematic of the samples imaged in FIGS. 15A and 15B.

FIG. 16A is an agarose gel characterization of antibodies anti-TGIT (lane 1) and anti-PD-1 (Lane) functionalized with dye-modified DNA.

FIG. 16B is a graph showing of the analytical SEC analysis of the samples in FIG. 16A.

FIG. 16C is a schematic of the model antibodies anti-TIGIT (Lane 1) and anti-PD-1 (Lane 4) were functionalized with dye-modified DNA (i.e., S2-DBCO-Cy3-S3, S4-DBCO-Cy5-S5, S6-DBCO-FITC-S1). The antibody-DNA conjugates (S2-anti-TIGIT-Cy3-S3 (Lanes 2, 3), S4-anti-PD-1-Cy5-S5 (Lanes 5, 6), and S6-anti-PD-1-FITC-S1 (Lanes 7, 8, 9)) were isolated and characterized using a. SDS PAGE (4-15%) and b. analytical SEC.

FIG. 17A is an agarose gel electrophoresis (1.5 v/v %) characterization of a trimer antibody-DNA oligomer assembled and purified using SEC. Fractions collected during the SEC purification were characterized and compared to antibody-DNA conjugates.

FIG. 17B is a graph showing the analytical SEC of the samples of FIG. 17A.

FIG. 17C is a schematic illustration of the samples characterized in FIGS. 17A and 17B.

FIG. 18A illustrates functionalization of the protein C-terminus with DNA via blue-light mediated photoredox reaction and subsequent strain-promoted azide-alkyne cycloaddition reaction with a DBCO-containing DNA strand.

FIG. 18B illustrates functionalization of the protein N-terminus with DNA via imine formation and irreversible cyclization reaction with 3-azido-2-pyridinecarboxaldehyde (A-2PCA) and subsequent strain-promoted azide-alkyne cycloaddition reaction with a DBCO-containing DNA strand.

FIG. 19A shows SDS-PAGE characterization of DNA conjugations targeting the C-terminus of model proteins, BSA (Lane 1) and GFP (Lane 4), were functionalized with a single DNA strand and BSA_{C-Term}-n-DNA (Lane 3) and GFP_{C-Term}-DNA (Lane 5) conjugates were isolated.

FIG. 19B shows SDS-PAGE characterization of DNA conjugations targeting the N-terminus of model proteins, BSA (Lane A), MBP (Lane C), and GOx (Lane E), were functionalized with a single DNA strand to prepare reaction mixtures containing BSA_{N-Term}-DNA (Lane B), MBP_{N-Term}-DNA (Lane D), and GOx_{N-Term}-DNA (Lane F).

FIG. 19C shows SDS-PAGE characterization of DNA conjugations targeting the single surface exposed free cysteine of BSA (Lane 1) was functionalized with a single DNA strand to prepare reaction mixtures containing BSA_{Cys}-DNA (Lane 2). This reaction mixture exhibited bands with distinct electrophoretic mobilities as compared to GFP_{C-Term}-DNA conjugates (Lane 3).

FIG. 20A is a schematic illustration of oligomerization GFP- and BSA-DNA conjugates into a trimer with predefined sequences. Oligomers were assembled using DNA-DNA interactions between antibody-DNA conjugates (i.e., S2-GFP_{C-Term}-53, S4-BSA_{Cys}-S5, S4-BSA_{C-Term}-S5, S6-BSA_{Cys}-S5, S6-BSA_{C-Term}-S1) and template DNA strands (i.e., S1′-S2′-T2′, S3′-54′-T4′, S5′-S6′-T6′).

FIG. 20B is a schematic illustration of oligomerization GFP- and BSA-DNA conjugates into a dimer with predefined sequences. Oligomers were assembled using DNA-DNA interactions between antibody-DNA conjugates (i.e., S2-GFP_{C-Term}-S3, S4-BSA_{Cys}-S5, S4-BSA_{C-Term}-S5, S6-BSA_{Cys}-S5, S6-BSA_{C-Term}-S1) and template DNA strands (i.e., S1′-S2′-T2′, S3′-54′-T4′, S5′-S6′-T6′).

FIG. 20C is a schematic illustration of oligomerization GFP- and BSA-DNA conjugates into a pentamer with predefined sequences. Oligomers were assembled using DNA-DNA interactions between antibody-DNA conjugates (i.e., S2-GFP_{C-Term}-S3, S4-BSA_{Cys}-S5, S4-BSA_{C-Term}-S5, S6-BSA_{Cys}-S5, S6-BSA_{C-Term}-S1) and template DNA strands (i.e., 51′-S2′-T2′, S3′-54′-T4′, S5′-S6′-T6′)

FIG. 21A is an Agarose gel electrophoresis of protein oligomers comparing the electrophoretic mobilities of template DNA strands (Lane 1), protein-DNA conjugates (Lane 2), a protein trimer (S2-GFP_{C-Term}-BSA_{Cys}-BSA_{C-Term}-S1, Lane 3), a protein dimer (S1′-GFP_{C-Term}-BSA_{C-Term}-S6′, Lane 4), a protein pentamer m-G (S2-GFP_{C-Term}-BSA_{Cys}-BSA_{C-Term}-GFP_{C-Term}-BSA_{C-Term}-S6′, Lane 5), fragmentation of the pentamer into trimers and monomers (removal of S3′-S4′-T4′, Lane 6), fragmentation of the pentamer into trimers and dimers (removal of S5′-S6′-T6′, Lane 7), and fragmentation of the pentamer into trimers and dimers (removal of S1′-S2′-T2′, Lane 8).

FIG. 21B is a scheme of the fragmentation pathways of a protein pentamer via toehold-mediated strand displacement reactions to confirm the sequence of oligomers. By removing template DNA strand 53′-54′-T4′, the pentamer fragments into monomers and trimers. In contrast, by removing either template DNA strand S5′-S6′-T6′ or S1′-52′-T2′, the pentamer fragments into dimers and trimers.

### DETAILED DESCRIPTION

Methods of the disclosure utilize native antibodies functionalized with a single DNA strand to from antibody-oligonucleotide conjugates which are then oligomerized with predefined sequences using a modular oligonucleotide scaffold. Antibody-oligonucleotides are also referenced herein as antibody-DNA conjugates. It should be understood, however, that any oligonucleotide including DNA and RNA can be used in the methods of the disclosure. Methods of the disclosure can provide a generalizable synthetic route to prepare sequence-encoded antibody oligomers.

Proteins are complex macromolecules with precise functions. However, the precise incorporation of proteins into materials is challenging. Methods of the disclosure provide a generalizable strategy to organize nearly any protein of interest into polymers that contain a predefined sequence, stoichiometry, and architecture. First, generalizable bioconjugation techniques are utilized to functionalize proteins with synthetic oligonucleotides, and protein-DNA conjugates that contain a single DNA modification are isolated. DNA-DNA interactions are used to assemble protein-DNA conjugates into polymers that contain a predefined sequence, number, and arrangement. This protein polymerization strategy enables the rapid preparation of libraries of molecularly defined protein-based materials.

There are greater than 170,000 known protein structures, with many exhibiting defined functions (e.g., catalysis, membrane transport, recognition).¹ Combining and repurposing proteins with different functions is proving to be a powerful approach for applications such as catalysts, materials, and therapeutics. However, there is not a generalizable method to conjugate proteins of interest to each other (i.e., polymerize them) without having to modify the native polypeptide sequences. The modular organization of proteins on the nanoscale could result in a nearly infinite number of multifunctional protein polymers based upon the structures and functions of the constituent proteins. Herein, we developed a synthetic strategy to organize nearly any protein or set of proteins into polymers with predefined sequences, stoichiometries, and architectures. To synthesize conjugates with a single oligonucleotide per protein, chemistry that is generalizable to nearly any protein (i.e., lysine-, cystine-, C-terminal, or N-terminal specific) was performed, and protein-DNA conjugates were isolated using standard protein purification techniques. Next, DNA-DNA interactions between protein-DNA conjugates and template DNA strands were used to assemble proteins into molecularly defined, sequence-specific protein polymers. This strategy enables the modular organization of proteins into polymers and the rapid preparation of libraries of protein polymers to be studied as catalysts, materials, or therapeutics.

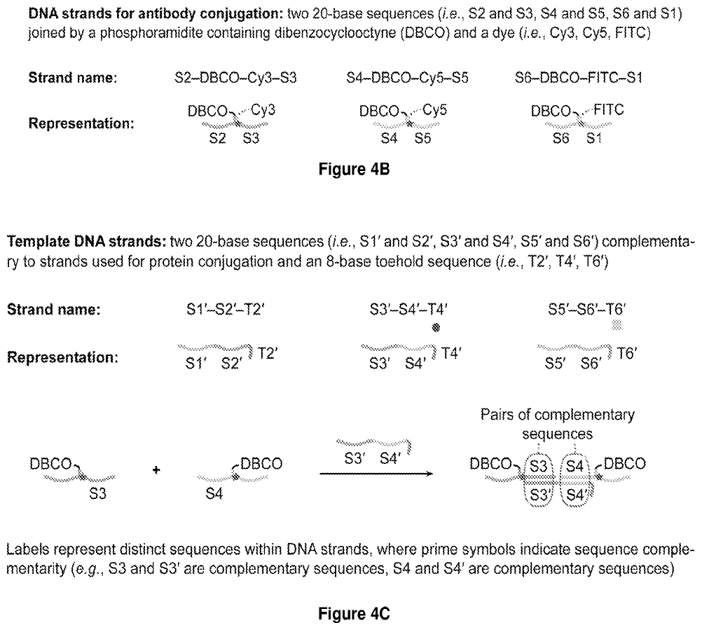

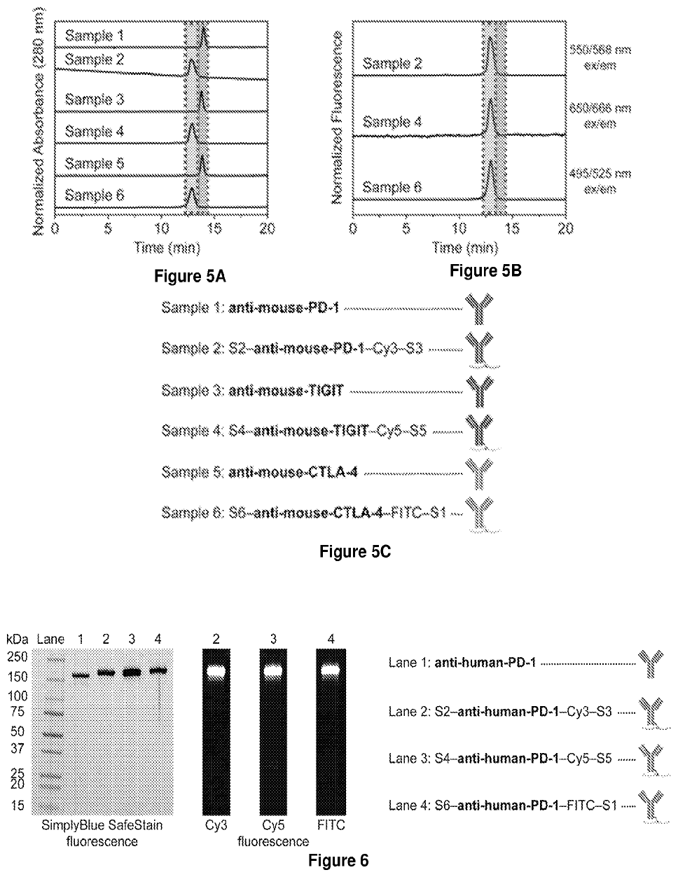

Methods of the disclosure can utilize modular DNA scaffolds designed and used to precisely organize antibodies into sequence-encoded oligomers, where both the number and sequence of antibodies can be controlled using DNA-DNA interactions. For example, in the examples presented herein, native antibodies were functionalized with a single DNA strand and, subsequently, antibody-DNA conjugates were oligomerized with predefined sequences using a modular DNA scaffold (FIG. 4, Tables 1 and 2, FIG. 5). This approach introduces a generalizable synthetic route to prepare sequence-encoded antibody oligomers. Overall, this advance can enable therapeutic developments of co-delivering multiple antibodies as single constructs with defined chemical architectures.

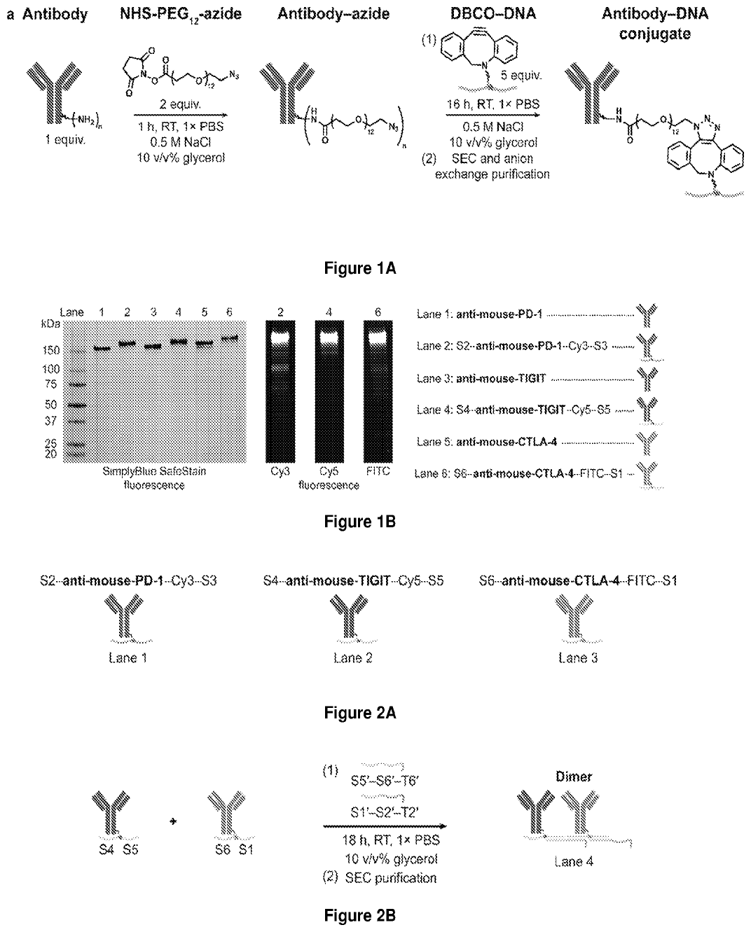

Referring to FIG. 1, antibody-oligonucleotide conjugates were formed by functionalizing primary amines on the surface of the antibody with azides. Any known chemistry to modify the antibody with an azide can be used. For example, NHS-PEG₁₂-azide linker can be used to functionalize the surface of the antibodies. Other linkers can include, for example, NHS-PEG₄-azide, NHS-azide, N-5-azido-2-nitrobenzoyloxysuccinimide, sulfosuccinimidyl-6-[4′-azido-2′-nitrophenylamino]hexanoate. Alkyne modified oligos can be conjugated to the azide-modified antibodies through a strain-promoted azide-alkyne cycloaddition. The oligos can be modified, for example, with dibenzocyclooctyne. Alternatively, the oligos could be modified with any other alkynes. The alkyne modified oligo includes the alkyne arranged between two distinct sequences. The distinct sequences allow for oligomerization of the antibody-oligonucleotide conjugate through hybridization of at least one of the distinct sequences to a template oligo.

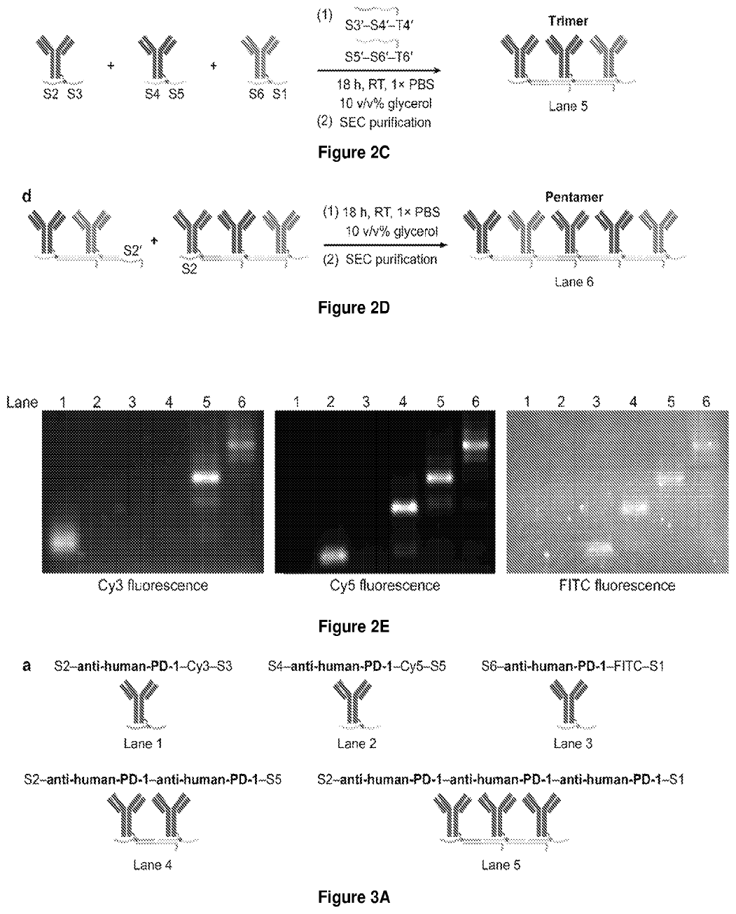

Referring to FIG. 2 and FIG. 4A, template oligos can be designed as complements to two sequences on different antibody-oligonucleotide conjugates. For example, a first antibody-oligonucleotide conjugate can have an alkyne modified oligo having first and second sequences with the alkyne arranged there between and a second antibody-oligonucleotide conjugate can have an alkyne modified oligo having third and fourth sequences with the alkyne arranged there between. The template oligo for oligomerization of the first and second antibody-oligonucleotide conjugates can be designed having at least first and second template sequences, the first template sequence being a complement of one of the first or second sequence and the second template sequence being a complement to one of the third or fourth sequence. That is, the template oligo is a complement of portions of the oligos of the first and second antibody-oligonucleotide conjugates such that the template oligo hybridizes to the complementary portions of the first and second antibody-oligonucleotide conjugates thereby oligomerizing the first and second antibody-oligonucleotide conjugates in a sequence encoded by the template oligo design.

Methods of the disclosure can include admixing any number of antibody-oligonucleotide conjugates with any number of templates to oligomerize the various antibody-oligonucleotides in a sequence encoded manner. For example, admixing first and second antibody-oligonucleotide conjugates with at least one template oligo under conditions sufficient to hybridize the at least one template oligo to the conjugates can result in formation of a dimer. For example admixing first, second, and third antibody-oligonucleotide conjugates with at least two template oligos under conditions sufficient to hybridize one of the at least two template oligos to portions of the first and second antibody-oligonucleotide conjugate and the other one of the at least two template oligos to portions of the second and third antibody-oligonucleotide or to the first and third antibody-oligonucleotide depending on the template design. Such a method results in formation of a trimer. In such methods, the at least two template oligos each have two template sequences and each of the template sequences of the at least two template oligos is a complement of distinct ones of the two linking sequences of the oligos of the antibody-oligonucleotides conjugates. As detailed above, each of the antibody-oligonucleotide conjugates has an oligo with two sequences arranged on opposed sides of an alkyne. For trimer formation, for example, the template oligos are designed such that a first template sequence of a first template oligo is complementary to a first linking sequence of a first antibody-oligonucleotide conjugate and a first template sequence of a second template oligo is complementary to a second linking sequence of the first antibody-oligonucleotide conjugate, with the second template sequence of the first template oligo being complementary to one of the linking sequences of a second antibody-oligonucleotide, and the second template sequence of the second template oligo being complementary to one of the linking sequences of a third antibody-oligonucleotide.

Dimers and trimers formed in accordance with methods of the disclosure can be further oligomerized using methods of the disclosure. For example, a dimer can be formed using two template oligos, such that the second template oligo has a first template sequence that hybridizes to a complementary linking sequence of one of the antibody-oligonucleotide conjugates and has a second template sequence that is not complementary to a linking sequence of either antibody-oligonucleotide in the dimer. This second template sequence can then define a dimer linking sequence. The trimer can be formed as described above, wherein at least one of the antibody-oligonucleotide conjugates has a linking sequence that is not hybridized to a template oligo, referred to herein as a free linking sequence. The dimer and trimer can be selected and designed such that the dimer linking sequence is complementary to the free linking sequence on the trimer. Alternatively, the dimer can have a free linking sequence and the trimer can be hybridized with a template oligo that has an unhybridized region defining a trimer linking sequence. In a still further alternative, template oligos with complementary template sequences to linking sequences of the dimer and trimer can be used.

### DNA System to Assemble Protein Polymers

A DNA system was designed to direct the assembly of protein-DNA conjugates into polymers that contain a predefined sequence, number, and arrangement of proteins (FIG. 1). In the designed DNA system, bolded labels represent distinct sequences within DNA strands (i.e., S1, S2, S3, S4, S5, S6, S1′, S2′, S3′, S4′, S5′, S6′, T2, T4, T6, T2′, T4′, T6′), and the complementarity of the sequences is indicated by the prime symbol (e.g., S1 and S1′ are complementary DNA sequences). DNA strands for protein conjugation were designed to contain two 20-base sequences (i.e., S2 and S3, S4 and S5, S6 and S1) joined by a phosphoramidite that contains dibenzocyclooctyne (DBCO, i.e., S2-DBCO-S3, S4-DBCO-S5, S6-DBCO-S1), which can undergo strain-promoted azide-alkyne cycloaddition (SPAAC) reactions with azide-functionalized proteins. Template DNA strands (i.e., S1′-S2′-T2′, S3′-S4′-T4′, S5′-S6′-T6) were designed with two 20-base sequences that are complementary to sequences within distinct protein-conjugated strands (i.e., S1′ and S2′, S3′ and S4′, S5′ and S6′), as well as an 8-base toehold overhang (i.e., T2′, T4′, T6′). The toehold facilitates the removal of template strands from protein polymers via strand displacement reactions with DNA strands that are fully complementary to the template strands (i.e., S1-S2-12, S3-S4-T4, S5-S6-T6). Each DNA strand was synthesized on solid supports and the masses of oligonucleotides were confirmed using matrix-assisted laser desorption ionization-time of flight mass spectrometry (MALDI-TOF MS). The sequence of each DNA strand described herein can be found in Table 1 below.

### Generalizable Synthesis of Antibody-DNA Conjugates

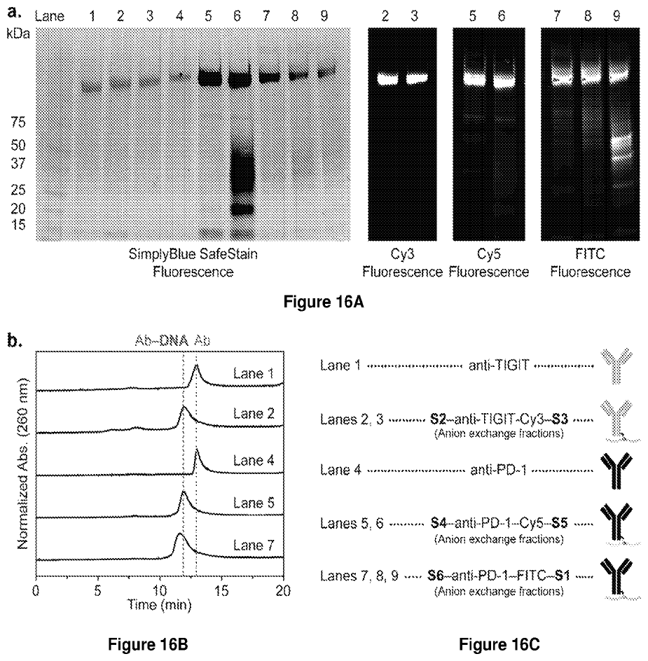

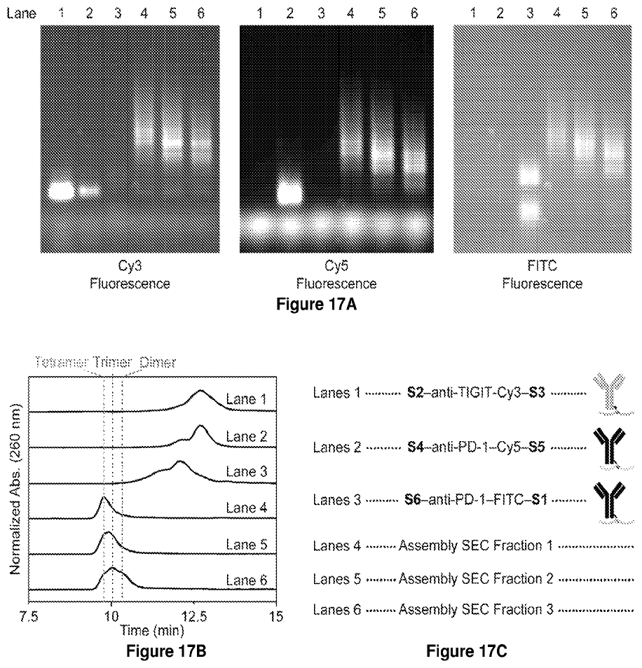

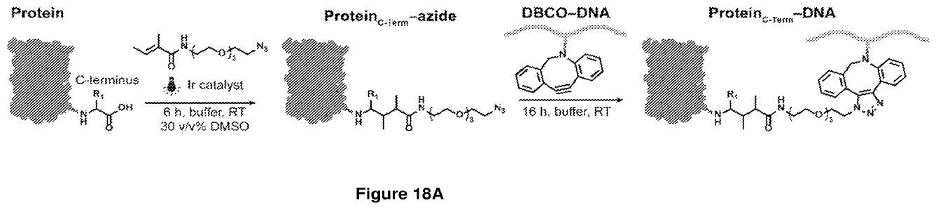

To synthesize antibody-DNA conjugates, lysine-targeting bioconjugation chemistry was used to install azides onto therapeutically-relevant antibodies (i.e., anti-mouse T cell immunoreceptor with Ig and ITIM domains (anti-TIGIT), anti-mouse programmed death-1 (anti-PD-1)) using azido-poly ethylene glycol-NHS ester, 2-3 where the extent of antibody functionalization can be readily controlled via reagent stoichiometry. Specifically, two equivalents of azido-poly ethylene glycol-NHS ester per antibody were mixed for 1 h at room temperature in 1×PBS containing 10% glycerol (v/v %, Scheme 1). The unreacted azido-poly ethylene glycol-NHS ester was removed from the reaction mixture using single-use size-exclusion columns (e.g., Cytiva NAP™ column, Thermo Scientific™ Zeba™ spin desalting column). Next, 10 equivalents of a DNA strand containing DBCO and a dye (i.e., S2-DBCO-Cy3-S3, S4-DBCO-Cy5-S5, S6-DBCO-FITC-S1) were added to conjugate DNA to the azido-modified protein using a SPAAC reaction (Scheme 1) for 16 h at room temperature in 1×PBS containing 10% glycerol (v/v %). Antibody-DNA conjugates with a single DNA modification per antibody were isolated using standard protein purification techniques (i.e., size-exclusion chromatography (SEC), anion exchange chromatography) and characterized using gel electrophoresis (FIG. 16A) and SEC (FIG. 16B).

Assembling Model Proteins into Molecularly Defined Oligomers

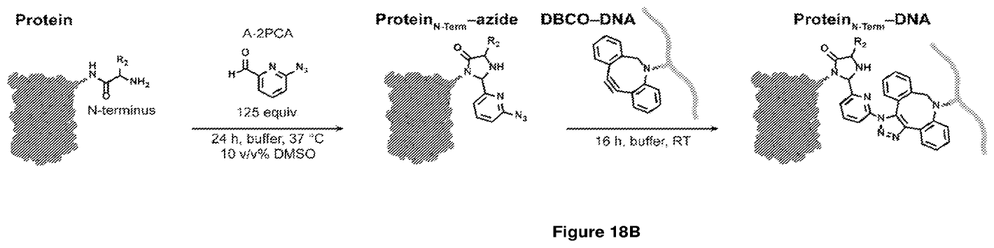

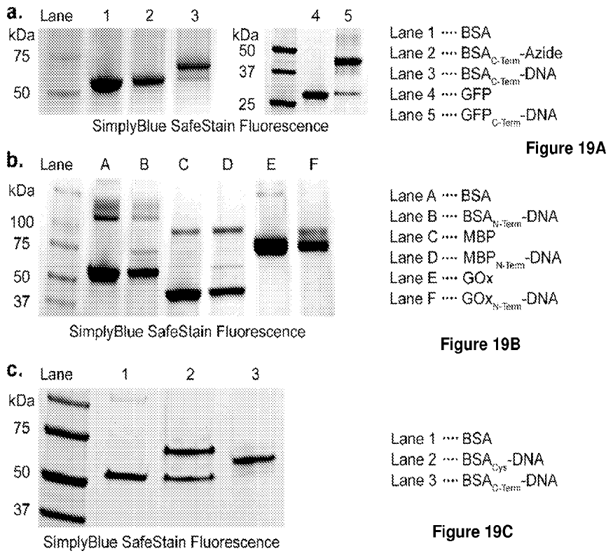

To generate protein-DNA conjugates where the location of DNA is predetermined, the C- and N-termini were targeted for bioconjugation because all monomeric proteins contain a single C-terminus and a single N-terminus. Furthermore, over 90% of protein termini are exposed to the solvent.⁴ However, due to the challenge of conjugating a single oligonucleotide to a multimeric protein (e.g., IgG antibodies), model monomeric proteins were evaluated. Green fluorescent protein (GFP), bovine serum albumin (BSA), maltose binding protein (MBP), and glucose oxidase (GOx) were employed for bioconjugation reactions targeting either the C- or N-terminus to install an azide. To functionalize the protein C-terminus with an azide, we modified reported photoredox reaction conditions where the C-terminal carboxylate is selectively oxidatively removed using an iridium photocatalyst.⁵ The C-terminal radical that results from decarboxylation can then add to an azide-containing Michael acceptor (FIG. 18A). Specifically, cis-dimethyl(N-9-ethylazide triethyleneglycol)acrylamide and (Ir[dF(CF3)ppy]2(dtbpy))PF6 were added to a buffer solution containing the model protein and 30% DMSO. The reaction mixture was purged for 10 min using Ar and subsequently illuminated with blue LEDs at room temperature for 6 h. To functionalize the protein N-terminus with an azide, the condensation reaction between the N-terminal amine and pyridinecarboxaldehyde6 was employed using 3-azido-2-pyridinecarboxaldehyde (A-2PCA, FIG. 18B). Specifically, A-2PCA was added in excess (125 equiv. relative to protein) and incubated at 37° C. for 24 h in a buffered solution containing 10% DMSO. To determine the success of the azide installation at either terminus, DBCO-DNA was added to proteins purified via SEC after functionalization. The resulting protein-DNA conjugate will show a change in electrophoretic mobility during SDS-PAGE analysis. Indeed, successful modifications of the C-terminus of BSA and GFP, as well as the N-terminus of BSA, MBP, and GOx with a single DNA strand were observed (FIGS. 19A and 19B, respectively). Following DNA-conjugation, the protein-DNA conjugates were readily purified using standard protein purification techniques (i.e., SEC, anion exchange chromatography) due to the increase in hydrodynamic size and change in surface charge. Finally, the purity of each protein-DNA conjugate was confirmed using SDS-PAGE.

In addition to the C-terminal and N-terminal functionalization, the single surface-exposed free cysteine of BSA was targeted for DNA bioconjugation. To synthesize the conjugate, amine-functionalized DNA was reacted with excess SPDP (50 equiv. relative to protein) for 45 min in 1×PBS at room temperature, then run through a NAP column to remove excess SPDP. Next, the amine-functionalized DNA was mixed with BSA in 1×PBS at room temperature overnight. Finally, the BSACys-DNA conjugates were purified using anion exchange chromatography and analyzed via SDS-PAGE to confirm a change in electrophoretic mobility (FIG. 19C).

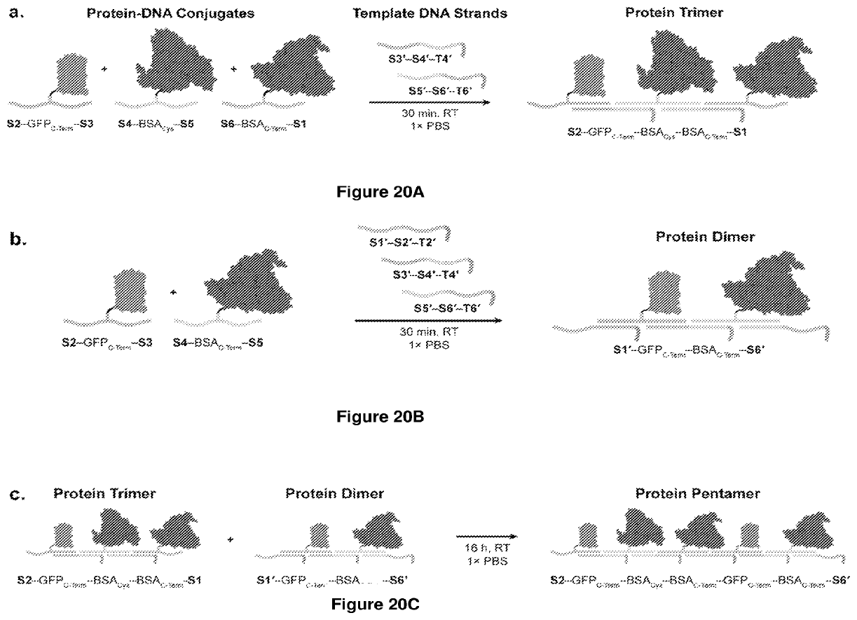

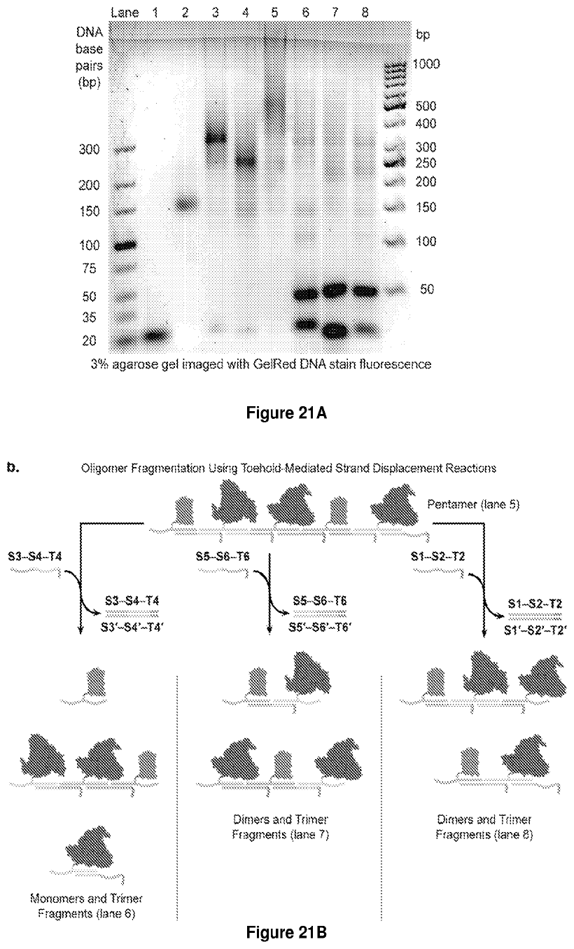

Next, dimers and trimers of the protein-DNA conjugates were synthesized. For the trimer, S2-GFPC-Term-S3, S4-BSACys-S5, and S6-BSAC-Term-S1 were mixed with two template DNA strands, S3′-S4′-T4′ and S5′-S6′-T6′ in stoichiometric amounts in 1×PBS (FIG. 20A). For the dimer, S2-GFPC-Term-S3 and S4-BSAC-Term-S5, were mixed with three template DNA strands, S1′-S2′-T2′, S3′-S4′-T4′, and S5′-S6′-T6′ in stoichiometric amounts in 1×PBS (FIG. 20B). After waiting 30 minutes, each assembly mixture was purified using SEC to isolate a sequence-encoded protein polymer (trimer: S2-GFPC-Term-BSACys-BSAC-Term-S1, dimer: S1′-GFPC-Term-BSAC-Term-S6′). These designs were selected because larger oligomers can be synthesized by combining the dimers and trimers with complementary single-stranded DNA sequences. For example, pentamers were synthesized by mixing the trimer S2-GFPC-Term-BSACys-BSAC-Term-S1 and the dimer S1′-GFPC-Term-BSAC-Term-S6′ in stoichiometric amounts in 1×PBS (FIG. 20C) and confirmed using agarose gel electrophoresis characterization (FIG. 21A, Lanes 1-5).

However, the agarose gel electrophoresis characterization data did not confirm the specific sequences of the protein-DNA oligomers. To characterize the oligomer sequence, the pentamer with an expected sequence of GFPC-Term-BSACys-BSAC-term-GFPC-Term-BSAC-Term was fragmented through the removal of template strands using toehold mediated strand displacement reactions where the fragmentation patterns after the removal depend on which strand is removed (FIG. 21B). Complements to the template strands (i.e., S1-S2-T2, S3-S4-T4, S5-S6-T6) were added to the pentamer in excess (2 equiv. relative to template strands) to remove the corresponding template strand. For example, S3-S4-T4 will preferentially bind to the S3′-S4′-T4′ template strand between both GFPC-term-BSACys and GFPC-Term-BSAC-Term due to the increased binding energy between the template strand and the complement resulting from the increased number of base pairs. If the sequence of protein-DNA oligomers is correct, a trimer and two monomers are expected upon template strand removal from the protein-DNA oligomer. Indeed, when analyzed by gel electrophoresis, bands corresponding to a trimer and monomers were observed (FIG. 21A, Lane 6). Importantly, a band was not observed that would correlate with a dimer in solution. Upon the addition of complementary strands that would yield a dimer and a trimer (either S5-S6-16 or S1-S2-T2), these products were also observed via agarose gel electrophoresis (FIG. 21A, Lanes 7 and 8, respectively). These results confirm that the model proteins could be oligomerized into sequence-defined supramolecular constructing via DNA-DNA interactions.

Here, DNA-DNA interactions are used to organize these proteins into polymers that contain a predefined sequence, number, and arrangement of proteins. This method is enabled by employing methods to functionalize nearly any protein with a single strand of DNA, and subsequently polymerizing the protein-DNA conjugates. This modular and generalizable approach introduces the ability to rapidly prepare libraries of sequence-encoded protein polymers.

This technology can enable the rapid synthesis of protein polymers that contain a predefined sequence, number, and arrangement of nearly any protein of interest without having to modify native protein sequences. Other techniques require recombinant protein expression to realize protein polymers with predefined sequence, number, and arrangement of proteins or lead to polymers without a predefined sequence, number, and arrangement of proteins.

In methods demonstrated herein native antibodies were functionalized with a single DNA strand and a modular DNA scaffold design was used to organize antibody-DNA conjugates into sequence-encoded antibody dimers, trimers, and pentamers where antibody function was retained. This modular synthetic route of the methods of the disclosure can enable investigations into oligomeric antibody constructs where the sequence, number, identity, and spatial location of antibodies within oligomers oligomer can be discretely modified in tandem with robust immunological investigations. Overall, chemical control over the location, valency, and variety of antibodies present in a single molecular construct can lead to materials that display emergent therapeutic efficacies.

### Examples

### Synthesis and Characterization of DNA

### Materials and Methods

Materials. All materials for oligonucleotide synthesis were obtained from Glen Research and were used as received.

High performance liquid chromatography (HPLC). Successfully synthesized oligonucleotides were isolated using reverse-phase HPLC on an Agilent Technologies 1260 Infinity II HPLC using an Agilent Dynamax Microsorb 300-10 C4 or Agilent Dynamax Microsorb 300-5 C18 column.

Matrix-assisted laser-desorption ionization time-of-flight mass spectrometry (MALDI-TOF MS). Successful synthesis of desired oligonucleotides was confirmed by MALDI-TOF MS. Samples were mixed with a 2′,6′-dihydroxyacetophenone matrix containing diammonium hydrogen citrate and characterized using a Bruker MALDI Rapiflex Tissue Typer instrument in linear mode with negative ion detection.

UV-Vis absorbance spectroscopy. Oligonucleotide concentrations were quantified by measuring the absorbance of oligonucleotide samples at 260 nm within the linear range on an Agilent Technologies Cary 60 UV-Vis spectrophotometer.

### Synthesis of Oligonucleotides

Oligonucleotides were synthesized on a 5 μmol scale on a Bio Automation MerMade 12 oligonucleotide synthesizer or an ABI 394 DNA synthesizer using standard solid phase synthetic protocols on controlled pore glass (CPG) beads. Successfully synthesized oligonucleotides were purified according to standard procedures, including HPLC. Successful synthesis of oligonucleotides was confirmed using MALDI-TOF MS and oligonucleotide concentration was quantified using UV-Vis absorbance spectroscopy at 260 nm.

DNA sequence design is provided in Table 1 below.

TABLE 1
Name, Sequences, Extinction Coefficients and Calculated and Observed
Molecular Weights of Oligonucleotides

| | | E₂₆₀ | MW (DA) | |
|---|---|---|---|---|
| SEQUENCE NAME | SEQUENCE (5′ TO 3′)^{A} | (M⁻¹ CM⁻¹)^{B} | CALCULATED^{B} | FOUND |
| S2-DBCO-CY3-S3^{C} | AACATCTTGTGCTCAATATC- | 406100 | 13485 | 13479 |
| | T(DBCO)-CY3- | | | |
| | TAGTCGTCTACGTAACAGTC | | | |
| | (SEQ ID NO: 1) | | | |
| S4-DBCO-CY5-S5^{C} | TTAGGCTGGATCTCGCGTTC- | 394400 | 13545 | 13563 |
| | T(DBCO)-CY5- | | | |
| | TGCACAGACCCATGTACTCG | | | |
| | (SEQ ID NO: 2) | | | |
| S6-DBCO-FITC-S1^{C} | TAACTCGTGAACGTATGCTC- | 407900 | 13571 | 13545 |
| | T(DBCO)-FITC- | | | |
| | AATCATCAGTACTCACCTAG | | | |
| | (SEQ ID NO: 3) | | | |
| S1′-S2′-T2′^{D} | GTAACGAT- | 488000 | 14925 | 14900 |
| | GATATTGAGCACAAGATGTT- | | | |
| | CTAGGTGAGTACTGATGATT | | | |
| | (SEQ ID NO: 4) | | | |
| S3′-S4′-T4′^{D} | CATTCAGA- | 479800 | 14779 | 14764 |
| | GAACGCGAGATCCAGCCTAA- | | | |
| | GACTGTTACGTAGACGACTA | | | |
| | (SEQ ID NO: 5) | | | |
| S5′-S6′-T6′^{D} | GTCATGTT- | 469700 | 14854 | 14832 |
| | GAGCATACGTTCACGAGTTA- | | | |
| | CGAGTACATGGGTCTGTGCA | | | |
| | (SEQ ID NO: 6) | | | |

^{A}DBCO-dT-CE, cyanine 3, cyanine 5, and fluoresceine phosphoramidites are represented by T(DBCO), Cy3, Cy5, and FITC, respectively.
^{B}Extinction coefficients and calculated molecular weights were calculated using the OligoAnalyzer Tool from IDT DNA.¹
^{C}Sequence names of DNA strands for antibody conjugation are listed in the 5′ to 3′ direction.
^{D}Sequence names of template DNA strands are listed in the 3′ to 5′ direction.

Table 2 provides the melting temperatures of complimentary DNA sequences within the designed oligonucleotides of Table 1. Melting temperatures were calculated using the OligoAnalyzer Tool from IDT DNA with DNA concentrations of 5 μm and salt concentrations from 1×PBS.

TABLE 2
Calculated Melting Temperatures

| DNA-DNA INTERACTION | T_{M} (° C.)^{A} |
|---|---|
| S1 AND S1′ | 60 |
| S2 AND S2′ | 60 |
| S3 AND S3′ | 62 |
| S4 AND S4′ | 68 |
| S5 AND S5′ | 68 |
| S6 AND S6′ | 64 |

Referring to FIG. 4A, three DNA strands were designed for conjugation to antibodies: S2-DBCO-Cy3-S3, S4-DBCO-Cy5-S5, and S6-DBCO-FITC-S1. Three DNA strands were designed to template the assembly of antibody-DNA conjugates-S1′-S2′-T2′, S3′-S4′-T4′, S5′-S6′-T6′. In the scheme of FIG. 4A, the symbols represent dye modifications to DNA strands in the scheme. In particular, the star represent Cy3, the circle represent Cy5, and the square represents FITC. Template DNA strands were designed to include toeholds (i.e., T2′, T4′, and T6′) to facilitate the removal of template DNA strands from oligomers via toehold-mediated strand displacement reactions, however, these reactions were not utilized. FIG. 4C shows DNA-DNA interactions between antibody conjugation strands and template strands used to organize antibodies that were functionalized with DNA into sequence-encoded oligomers. Labels on the DNA strands represent distinct sequences and pairs of sequences that have and lack the prime symbol are complementary and will interaction specifically (e.g., S3 and S3′ are complementary sequences, S4 and S4′ are complementary sequences. Complementary sequences were designed with melting temperatures between 60° C. and 70° C. in 1×PBS to facilitate assembly at room temperature at micromolar concentrations. Table 2 shows the calculation of melting temperatures.

### Synthesis and Characterization of Antibody-DNA Conjugates

Materials. InVivoMAb anti-mouse-PD-1 (CD279, clone RMP1-14), InVivoMAb anti-mouse-TIGIT (clone 1G9), InVivoMAb anti-mouse-CTLA-4 (CD152, clone 9H10), and InVivoSIM anti-human-PD-1 (Nivolumab Biosimilar) were obtained from Bio X Cell and used as received. Azido-dPEG®₁₂-NHS ester was obtained from Quanta Biodesign and used as received.

Preparative size-exclusion chromatography (SEC). The volumes of antibody-DNA conjugation reaction mixtures were reduced to ˜300 μL using Amicon Ultra centrifugal filters with a 30 kDa cutoff. Next, samples were purified on a Bio-Rad ENrich™ SEC 650 column on a Bio-Rad NGC Quest 10 Plus Chromatography System. SEC purification was performed in 10 mM TRIS:HCl (pH 7.4) buffer containing 500 mM NaCl at a flow rate of 0.33 mL/min.

Preparative anion exchange chromatography. Antibody-DNA conjugation reaction mixtures were diluted to ˜50 mL in 10 mM TRIS:HCl (pH 7.4) buffer containing 10 mM NaCl. Next, samples were purified by anion exchange on a Bio-Rad NGC Quest 10 Plus Chromatography System. Samples were loaded at a flow rate of 4.0 mL/min onto a column packed with Macro-Prep DEAE resin that was obtained from Bio-Rad. Antibodies were eluted from the column with 10 mM TRIS:HCl (pH 7.4) buffer containing 200 mM NaCl at a flow rate of 5.0 mL/min. Antibody-DNA conjugates were eluted from the column using a 20 minute gradient from 10 mM TRIS:HCl (pH 7.4) buffer containing 400 mM NaCl to 10 mM TRIS:HCl (pH 7.4) buffer containing 800 mM NaCl at a flow rate of 5.0 mL/min.

Analytical sodium dodecyl sulfate polyacrylamide gel electrophoresis (SDS PAGE). Samples were diluted with Bio-Rad Laemmi buffer and heated to 85° C. for 5 minutes. Next, samples were loaded into Bio-Rad Mini-Protean® TGX™ precast SDS PAGE gels and gels were run in 1× Tris/Glycine/SDS buffer from Bio-Rad for 35 minutes at 200 V. Gels were washed three times with heated water and imaged for Cy3, Cy5, and FITC fluorescence on a Bio-Rad ChemiDoc MP Imaging System. Gels were then stained with Thermo Fisher Scientific SimplyBlue™ SafeStain and imaged for SimplyBlue™ SafeStain fluorescence on a Bio-Rad ChemiDoc MP Imaging System.

Analytical SEC. Antibody and antibody-DNA conjugate samples were characterized by analytical SEC on an Agilent Technologies 1260 Infinity HPLC. Samples were run through an AdvanceBio SEC 300 Å 2.7 μm column in 1×PBS (pH 7.4) at a flow rate of 0.5 mL/min. Sample elution from the SEC column was tracked by measuring absorbance at 280 nm and fluorescence of dye modifications (i.e., Cy3 at 550/568 nm excitation (ex)/emission (em), Cy5 at 650/666 nm ex/em, FITC at 495/525 nm ex/em).

UV-Vis absorbance spectroscopy. Antibody-DNA conjugate concentrations were quantified by measuring the absorbance of dye modifications (i.e., Cy3 at 550 nm, Cy5 at 650 nm, FITC at 495 nm) within the linear range on an Agilent Technologies Cary 60 UV-Vis spectrophotometer.

### Synthesis of Antibody-DNA Conjugates

The co-administration of different combinations of antibody checkpoint inhibitors has been shown to have synergistic efficacy in vivo.⁸⁻¹⁰ Therefore, the mouse antibody checkpoint inhibitors anti-mouse-PD-1, anti-mouse-TIGIT, and anti-mouse-CTLA-4 were selected for the proof-of-concept demonstration of sequence-encoded antibody oligomerization using DNA. To install a single DNA strand onto antibodies (i.e., anti-mouse-PD-1, anti-mouse-TIGIT, anti-mouse-CTLA-4, anti-human-PD-1), one equivalent of antibody was added to two equivalents of a molecule containing an N-hydroxysuccinimide activated ester and an azide (NHS-PEG₁₂-azide) in 10 mM TRIS:HCl (pH 7.4) buffer containing 500 mM NaCl and 10% (v/v) glycerol.² The reaction was allowed to proceed for 45 min at room temperature. Next, excess unreacted NHS-PEG₁₂-azide ester was removed from the reaction mixture using a Cytiva NAP DNA purification column that was equilibrated in 10 mM TRIS:HCl (pH 7.4) buffer containing 500 mM NaCl and 10% (v/v) glycerol. To functionalize azide-modified antibodies with DNA, one equivalent of azide-modified antibodies was added to five equivalents of DBCO-modified DNA (DNA strands for antibody conjugation, i.e., S2-DBCO-Cy3-S3, S4-DBCO-Cy5-S5, S6-DBCO-FITC-S1) and the resulting strain promoted azide-alkyne cycloaddition was allowed to proceed for 16 h at room temperature. Excess unreacted DNA was removed from the reaction mixture using two successive rounds of preparative SEC and complete removal of DNA was confirmed using analytical SDS PAGE. Excess unreacted antibodies or antibodies functionalized with multiple DNA strands were removed using preparative anion exchange chromatography. Analytical SDS PAGE and analytical SEC were used to confirm the isolation of antibodies that were functionalized with a single DNA strand. Antibody-DNA conjugate samples were exchanged four times into 1×PBS (pH 7.4) buffer containing 10% (v/v) glycerol using Amicon Ultra centrifugal filters with a 30 kDa cutoff. Antibody-DNA conjugate concentrations were quantified using UV-Vis absorbance spectroscopy by measuring the absorbance of dye modifications (i.e., Cy3, Cy5, FITC).

This chemistry targets the primary amines (e.g., ε-amines on lysines, α-amines on N-termini)⁵⁸ on both the Fc and Fab regions of the antibody. The number of azide modifications per antibody were controlled by the amount of NHS-PEG₁₂-N₃ added (FIG. 1A). After purification by size-exclusion chromatography (SEC), the azide on the surface of each antibody underwent a strain-promoted azide-alkyne cycloaddition reaction with 5 equiv. of DNA strands containing dibenzocyclooctyne (DBCO) and fluorophore (i.e., Cy3, Cy5, FITC) between two distinct 20 base sequences (S2-DBCO-Cy3-S3, S4-DBCO-Cy5-S5, or S6-DBCO-FITC-S1, Table 1). After 16 h, antibodies that were functionalized with a single DNA strand were isolated by using SEC to remove unreacted DNA, and anion exchange chromatography to remove unreacted antibodies and antibodies that were functionalized with multiple DNA strands (FIG. 1a). Three different conjugates (i.e., S2-anti-mouse-PD-1-Cy3-S3, 54-anti-mouse-TIGIT-Cy5-S5, and S6-anti-mouse-CTLA-4-FITC-S1) were attained and confirmed to contain a single DNA functionalization via SDS-PAGE (FIG. 1B) and SEC (FIG. 5)

Referring to FIG. 5, analytical SEC characterization of mound antibody-DN conjugation was performed. FIG. 5A shows normalized absorbance at 280 nm, while FIG. 5B shows fluorescence of DNA-conjugated fluorophores. In both graphs, the elution times from 12.25-13.50 min are highlighted with a box bordered with squares and the elution times from 13.50-14.30 are highlighted with a box bordered with circles. FIG. 5C is an identification of the samples shown in FIGS. 5A and 5B.

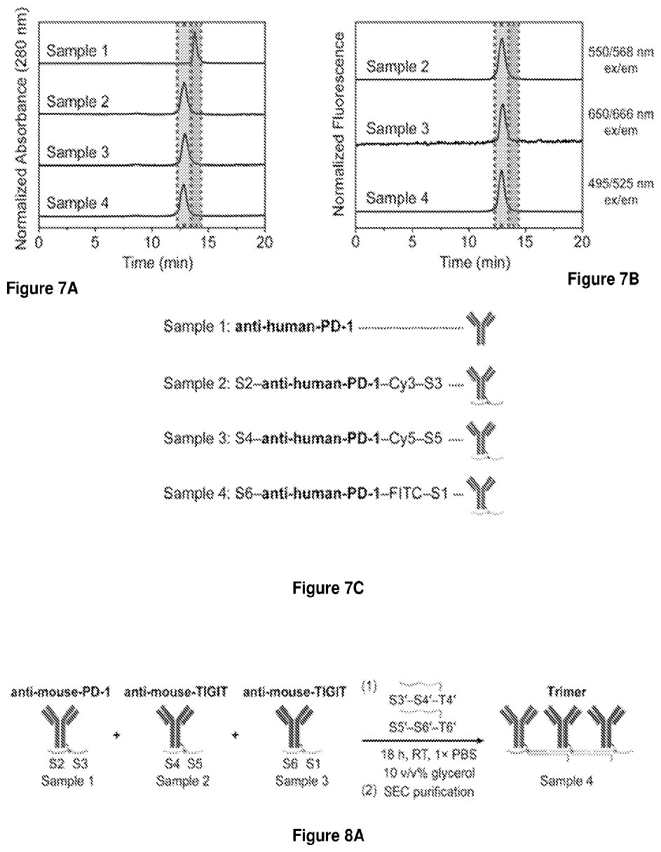

Referring to FIG. 6, human antibody functionalized with a single DNA strand was characterized by SDS PAGE. A single gel was imaged for SimplyBlue SafeStain, Cy3, Cy5, and FITC fluorescence. The human antibody functionalized with a single DNA strand was also characterized by analytical SEC page. FIG. 7A shows absorbance at 280 nm and FIG. 7B shows fluorescence of the DNA-conjugated fluorophores. In both graphs, the elution times from 12.25-13.50 min are highlighted with a box bordered with squares and the elution times from 13.50-14.30 min are highlighted with a box bordered with circles. FIG. 7C is an identification of the samples.

### Synthesis and Characterization of Sequence-Encoded Antibody Oligomers

Preparative SEC. The volumes of antibody oligomer assembly mixtures were reduced to ˜300 μL using Amicon Ultra centrifugal filters with a 30 kDa cutoff. Next, samples were purified on a GE Healthcare Superose™ 6 Increase 10/300 GL column on a Bio-Rad NGC Quest 10 Plus Chromatography System. SEC purification was performed in 1×PBS (pH 7.4) buffer at a flow rate of 0.20 mL/min.

Analytical agarose gel electrophoresis. Gels were cast containing 2% (w/w) agarose in 1× Tris-acetate buffer containing SDS from ThermoFisher Scientific. Next, samples were loaded into gels and the gels were run in 1× Tris-acetate buffer containing SDS for 90 minutes at 120 V. Gels were imaged for Cy3, Cy5, and FITC fluorescence on a Bio-Rad ChemiDoc MP Imaging System.

Analytical SEC. Antibody-DNA conjugate and antibody oligomer samples were characterized by analytical SEC on an Agilent Technologies 1260 Infinity HPLC. Samples were run through an AdvanceBio SEC 300 Å 2.7 μm column in 1×PBS (pH 7.4) at a flow rate of 0.5 mL/min. Sample elution from the SEC column was tracked by measuring absorbance at 280 nm and fluorescence of dye modifications (i.e., Cy3 at 550/568 nm excitation ex/em, Cy5 at 650/666 nm ex/em, FITC at 495/525 nm ex/em).

UV-Vis absorbance spectroscopy. Antibody oligomer concentrations were quantified by measuring the absorbance of dye modifications (i.e., Cy3 at 550 nm, Cy5 at 650 nm, FITC at 495 nm) within the linear range on an Agilent Technologies Cary 60 UV-Vis spectrophotometer.

### Synthesis of Sequence-Encoded Antibody Oligomers

Antibody oligomers were synthesized by mixing the purified antibody-DNA conjugates (FIG. 2a, 2e—Lanes 1-3) with template DNA strands. To assemble antibody-DNA conjugates into antibody dimers and sequence-encoded antibody trimers, equimolar antibody-DNA conjugates and template DNA strands with complementary DNA sequences were mixed in 1×PBS (pH 7.4) buffer containing 10% (v/v) glycerol. The assembly was allowed to proceed for 1 h at room temperature. Next, antibody dimers and sequence-encoded antibody trimers were isolated using preparative SEC and exchanged four times into 1×PBS (pH 7.4) buffer containing 10% (v/v) glycerol. Oligomer concentrations were quantified using UV-Vis absorbance spectroscopy by measuring the absorbance of Cy5 dye modifications. Analytical agarose gel electrophoresis and analytical SEC were used to confirm the isolation of pure antibody dimers and sequence-encoded antibody trimers. Antibody dimers and sequence-encoded antibody trimers with complementary single stranded DNA sequences were mixed in 1×PBS (pH 7.4) buffer containing 10% (v/v) glycerol to target the assembly of sequence-encoded antibody pentamers. The assembly was allowed to proceed for 18 h at room temperature. Next, sequence-encoded antibody pentamers were isolated using preparative SEC and exchanged four times into 1×PBS (pH 7.4) buffer containing 10% (v/v) glycerol. Analytical agarose gel electrophoresis and analytical SEC were used to confirm the isolation of pure sequence-encoded antibody pentamers.

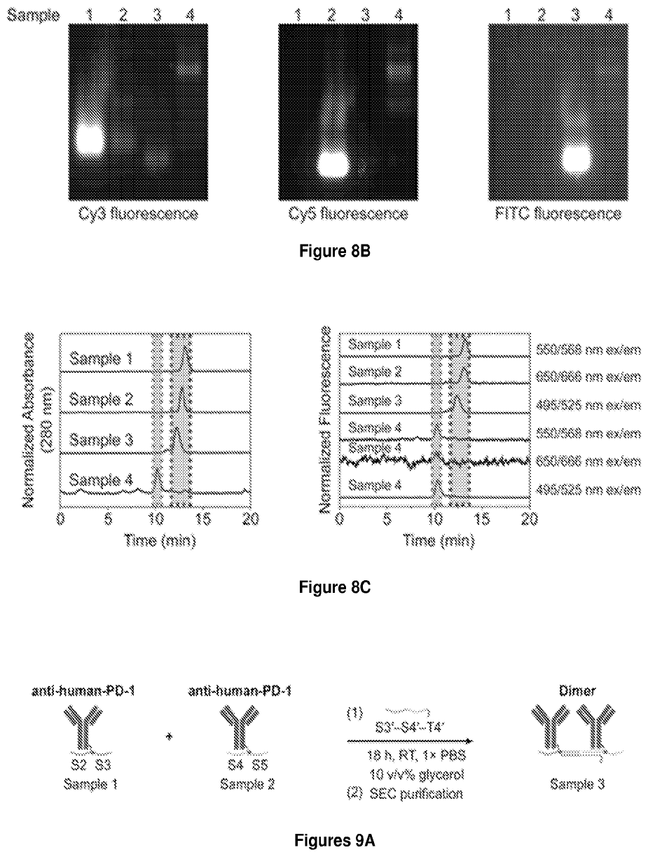

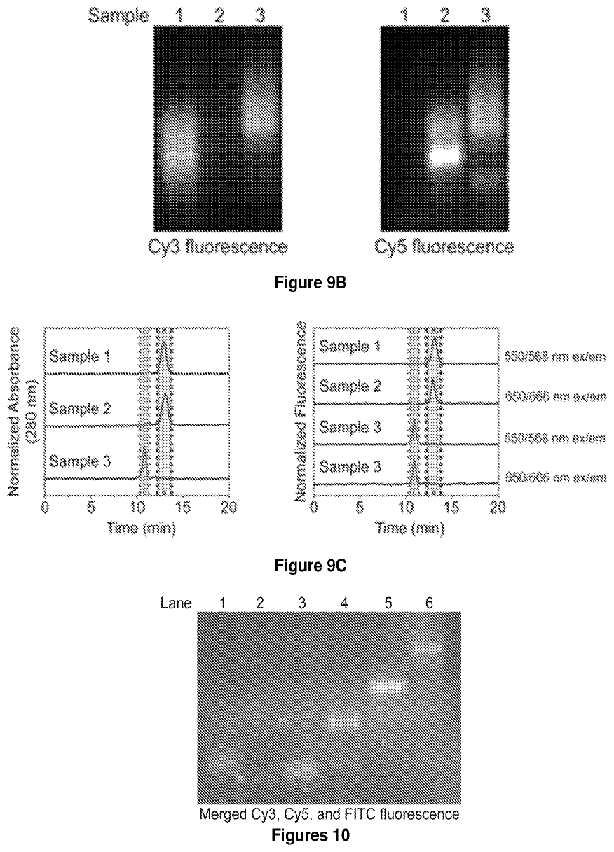

The template strands were designed as complements to two 20 base sequences on different antibody-DNA conjugates (Tables S1 and S2, Scheme S1). For example, the S5′ sequence on the template strand S5′-56′-T6′ is complementary to the S5 sequence on S4-anti-mouse-TIGIT-Cy5-S5 and the S6′ sequence is complementary to the S6 sequence on S6-anti-mouse-CTLA-4-FITC-S1. An S4-anti-mouse-TIGIT-anti-mouse-CTLA-4-S2′ antibody dimer was successfully synthesized and purified using equal amounts of the anti-mouse-TIGIT-DNA conjugate, anti-mouse-CTLA-4-DNA conjugate, S5′-S6′-T6′ template strand, and S1′-52′-T2′ template strand (FIG. 2b, 2e—Lane 4). Furthermore, a sequence-encoded S2-anti-mouse-PD-1-anti-mouse-TIGIT-anti-mouse-CTLA-4-S1 trimer was successfully synthesized and purified by mixing equal amounts of the anti-mouse-PD-1-DNA conjugate, anti-mouse-TIGIT-DNA conjugate, anti-mouse-CTLA-4-DNA conjugate with the template strands S3′-54′-T4′ and S5′-S6′-T6′ (FIG. 2c, 2e—Lane 5). Importantly, the dimer showed only the expected Cy5 and FITC fluorescence with lower electrophoretic mobility than either antibody-DNA conjugate alone. Additionally, the trimer showed the expected Cy3, Cy5, and FITC dye fluorescence, with lower electrophoretic mobility than the dimer. To ensure that this synthetic technique is generalizable, dimers and trimers containing different sequences of antibodies were also synthesized (FIGS. 8 and 9). Furthermore, analytical SEC analysis of antibody oligomers showed decreases in retention time with increases in the degree of oligomerization and corroborated the monodispersity of each oligomer assembly (FIGS. 8 and 9). These results confirm that both antibody dimers and sequence-encoded antibody trimers can be synthesized using antibody-DNA conjugates and the designed DNA scaffold.

To understand whether this DNA scaffold enables larger oligomers to be synthesized beyond trimers, a sequence-encoded S4-anti-mouse-TIGIT-anti-mouse-CTLA-4-anti-mouse-PD-1-anti-mouse-TIGIT-anti-mouse-CTLA-4-S1 antibody pentamer was synthesized by mixing the S4-anti-mouse-TIGIT-anti-mouse-CTLA-4-S2′ dimer and S2-anti-mouse-PD-1-anti-mouse-TIGIT-anti-mouse-CTLA-4-S1 trimer together at a 1:1 ratio (FIG. 2D). After SEC purification, a monodisperse pentamer construct was observed by agarose gel electrophoresis (FIG. 2e—Lane 6). These results demonstrate the modularity of the designed DNA scaffold to achieve oligomers with distinct sequences and numbers of antibodies higher than have been previously achieved using other DNA assembly, recombinant protein expression, or chemical conjugation approaches.

Oligomerization of mouse antibody-DNA trimer was characterized with agarose gel and analytical SEC. FIG. 8A illustrates the assembly scheme. Mouse antibody-DNA conjugates were assembled into a mouse antibody trimer using DNA-DNA interactions. FIG. 8B shows the agarose gel characterization of mouse antibody-DNA conjugates and a mouse antibody trimer. A single gel was imaged for Cy3, Cy5, and FITC fluorescence. FIG. 8C shows the analytical SEC characterization using absorbance at 280 nm and fluorescence of DNA-conjugated fluorophores. In both graphs, the elution times from 9.75-10.60 min and 11.80-13.60 min are highlighted.

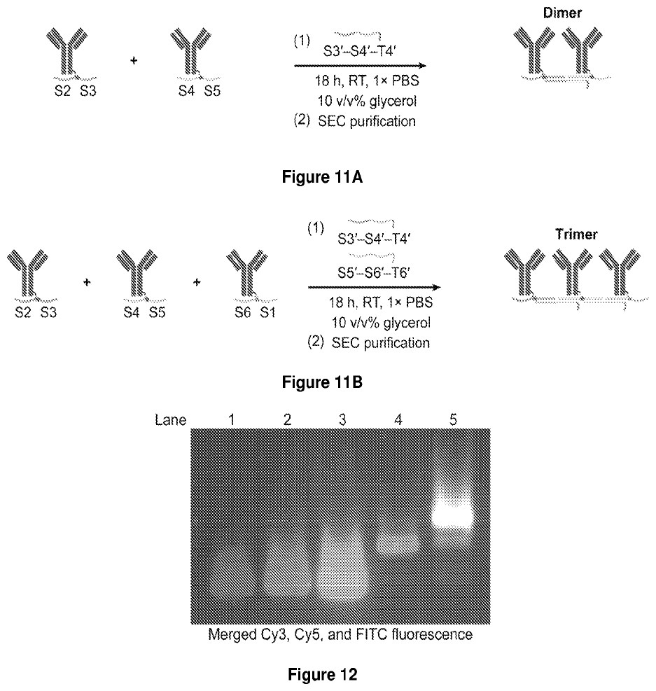

FIG. 11A shows the assembly of human antibody-DNA conjugates into a human antibody dimer. FIG. 11B shows the assembly of human antibody-DNA conjugates into a human antibody-DNA trimer. Oligomerization of human antibody-DNA dimer was characterized with agarose gel and analytical SEC. FIG. 9A illustrates the assembly scheme. Human antibody-DNA conjugates were assembled into a human antibody dimer using DNA-DNA interactions. FIG. 9B shows the agarose gel characterization of human antibody-DNA conjugates and a human antibody dimer. A single gel was imaged for Cy3, Cy5, and FITC fluorescence. FIG. 9C shows the analytical SEC characterization using absorbance at 280 nm and fluorescence of DNA-conjugated fluorophores. In both graphs, the elution times from 10.30-11.40 min and 12.20-13.80 min are highlighted.

FIG. 10 shows a merged agarose gel image of mouse antibody-DNA oligomers showing mouse antibody-DNA conjugates, a mouse antibody dimer, a mouse antibody trimer, and a mouse antibody pentamer. A single gel was imaged for Cy3, Cy5, and FITC fluorescence. The Cy3, Cy5, and FITC images shown in FIG. 2E were merged into a composite image in FIG. 10.

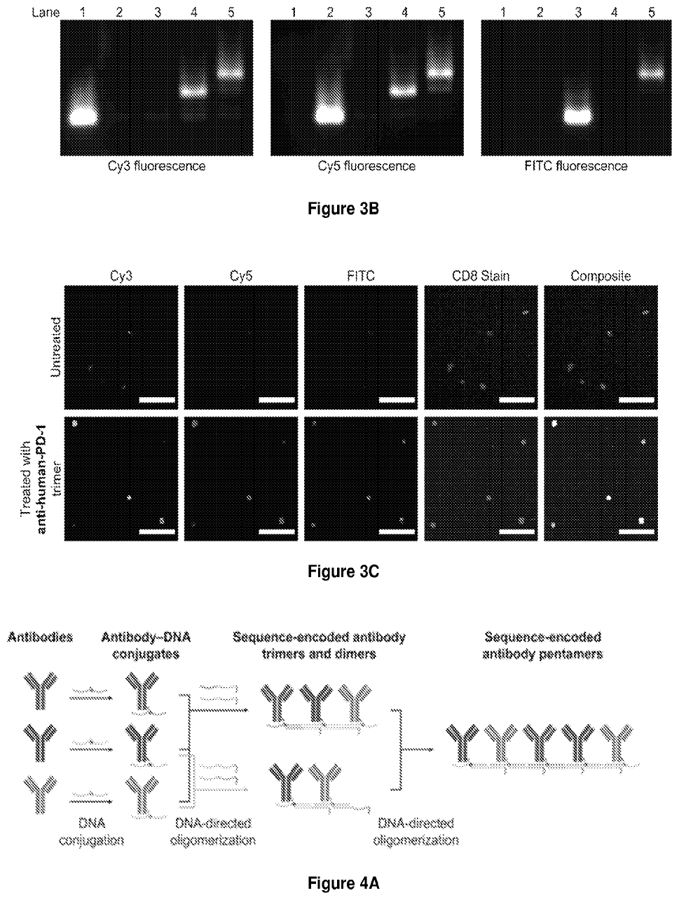

FIG. 12 shows a merged agarose gel image of human antibody-DNA oligomers showing human antibody-DNA conjugates, a human antibody dimer, and a human anybody trimer. A single gel was imaged for Cy3, Cy5, and FITC fluorescence. The Cy3, Cy5, and FITC images of FIG. 3B were merged into one composite image in FIG. 12.

### In Vitro Experiments to Measure Antibody Oligomer Binding to Cells and Efficacy as Checkpoint Inhibitors

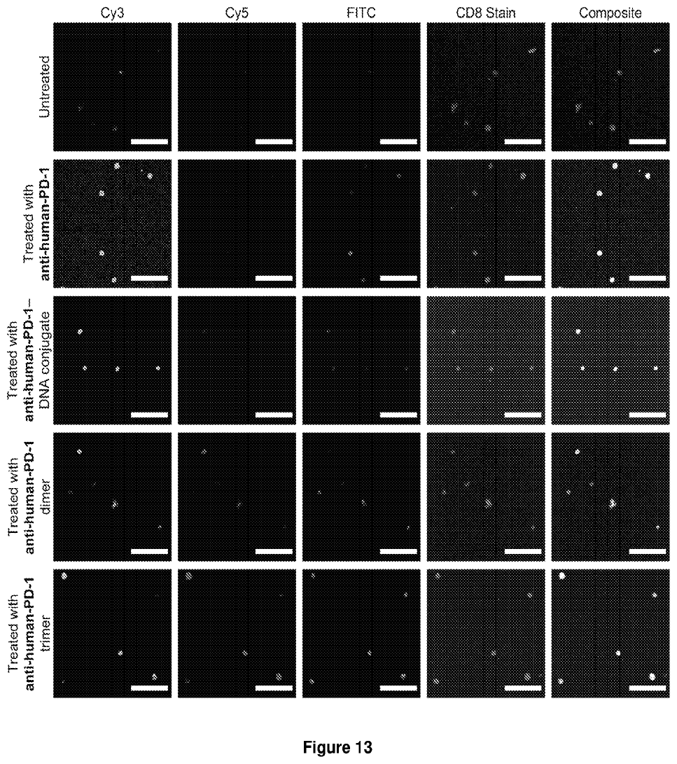

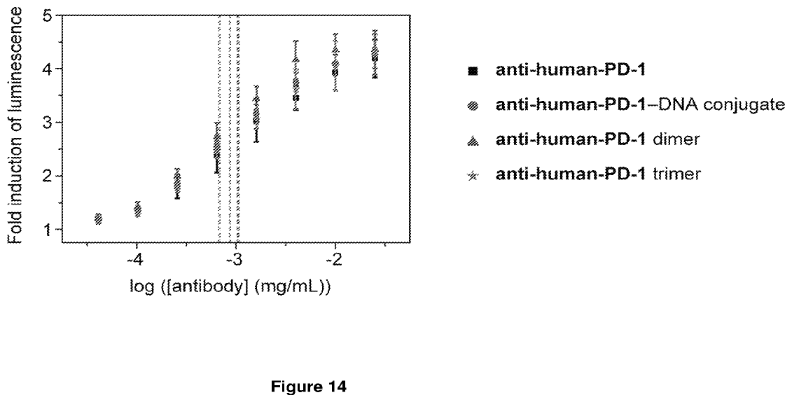

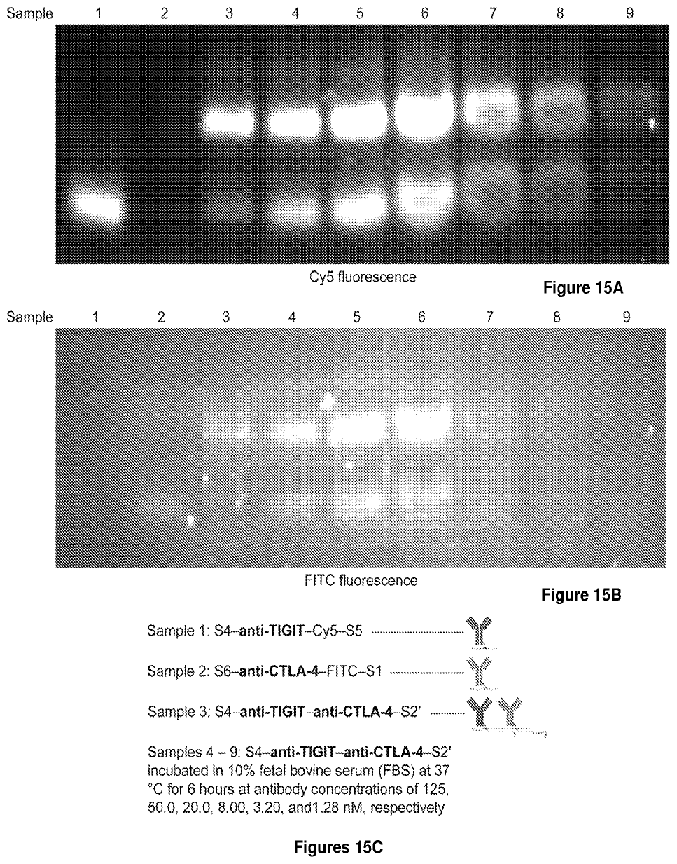

To ensure that checkpoint inhibitors retained their function following DNA conjugation and oligomerization, in vitro experiments were performed using a human antibody checkpoint inhibitor, anti-human-PD-1. First, anti-human-PD-1 was functionalized with the designed DNA strands (FIGS. 6 and 7). Subsequently, anti-human-PD-1-DNA conjugates were oligomerized using the template DNA strands (FIG. 11) to synthesize pure anti-human-PD-1-anti-human-PD-1 dimers (FIG. 3A—Lane 4) and anti-human-PD-1-anti-human-PD-1-anti-human-PD-1 trimers (FIG. 3A—Lane 5). Oligomers at total antibody concentrations of 50 nM were incubated with human peripheral blood mononuclear cells (hPBMCs) stimulated to overexpress PD-1 for 6 h at 37° C., then fixed and mounted for imaging. Confocal microscopy imaging of the cells showed the presence of Cy3, Cy5, and FITC fluorescence corresponding to the fluorophores expected from anti-human-PD-1-anti-human-PD-1-anti-human-PD-1 trimers when compared to an untreated control (FIG. 3C), as well as when compared to anti-human-PD-1, anti-human-PD-1-DNA conjugate, or anti-human-PD-1-anti-human-PD-1 dimer samples (FIG. 13). These results confirmed that the antibodies retain their ability to bind PD-1 checkpoint proteins following oligomerization. In addition to checkpoint protein recognition, an engineered luciferase reporter cell assay was used to test whether oligomerized anti-human-PD-1 antibodies can still function as checkpoint inhibitors. Importantly, the oligomers retained function over concentrations from 0.273 nM to 167 nM and the dimer showed a modestly lower EC50 compared to the unmodified anti-human-PD-1 (FIG. 14). To understand the oligomer stability at these treatment concentrations, a dimer of anti-mouse-CLTA-4 and anti-mouse-TIGIT was incubated in PBS containing 10% FBS at concentrations from 125-1.28 nM to for 6 h at 37° C., then analyzed using agarose gel electrophoresis. Densitometry analysis of images of the agarose gels revealed minimal degradation, as the purity of the dimer sample decreased from 85% dimer to 70% dimer regardless of sample concentration (FIG. 15). While studying the therapeutic implications of checkpoint inhibiting antibody oligomers is challenging without a full immune system, these results are important first steps towards understanding if antibody oligomers show emergent properties compared to unmodified antibodies.

Antibody binding assay. Human peripheral blood mononuclear cells (hPBMCs, Zenbio, SER-PBMC-P-F) were taken from a liquid nitrogen dewar and thawed in a water bath. Next, cells were added to 9 mL of RPMI media containing 10% (v/v) heat-inactivated fetal bovine serum and 1% (w/v) penicillin-streptomycin (herein termed RPMI+/+media). Cells were pelleted by centrifugation at 300×g for 10 min. The supernatant was removed by aspiration and cells were resuspended in 3 mL of RPMI+/+media. Cells were counted using a Vi-CELL BLU Cell Viability Analyzer. Cells were adjusted to a concentration of 1×10⁶ cells/mL through dilution with RPMI+/+media containing 20 ng/mL phorbol myristate acetate (PMA) and 1000 ng/mL ionomycin to stimulate overexpression of PD-1.³⁻⁴ A volume of 200 μL of the diluted cells was added to each well of a 96-well plate with round bottom wells. Plates containing cells were incubated at 37° C. with 5% CO 2 for 48 h. After incubation, cells and solution were transferred from each well into flow inserts and washed with RPMI+1+media. Flow inserts were centrifuged at 1200 rpm for 5 min and samples were aspirated and resuspended in 200 μL of treatment. Treatment involved the addition of anti-human-PD-1, anti-human-PD-1-DNA conjugate (i.e., S2-anti-human-PD-1-Cy3-S3), anti-human-PD-1 dimer (i.e., S2-anti-human-PD-1-anti-human-PD-1-S5), or anti-human-PD-1 trimer (i.e., S2-anti-human-PD-1-anti-human-PD-1-anti-human-PD-1-S1) in RPMI+/+media at an antibody concentration of 50.0 nM. Samples were then incubated at 37° C. with 5% CO₂ for 6 h. After incubation, samples were washed once with 1×PBS (pH 7.4) buffer and resuspended in 100 μL 1×PBS (pH 7.4) buffer containing 0.5 μL of a BV421 CD8 antibody stain (Biolegend clone RPA-T8 #301036). Cells were incubated for 15 min at 4° C. Next, samples were washed once with 1×PBS (pH 7.4) buffer and resuspended in 100 μL fixation buffer (Biolegend #420801). Samples were stored at 4° C. prior to analysis.

Confocal microscopy. After treated hPBMC samples were stained and fixed, the samples were centrifuged at 300×g for 5 min and cell concentration was adjusted to 2×10⁶ cells/mL. A volume of 50 μL of each sample was mounted onto microscopy slides using ProLong Glass Anti-Fade Mountant (Invitrogen, #P36984) and allowed to cure overnight. Fluorescent confocal microscopy was performed using a Zeiss LSM800 microscope (40× objective, GaAsP PMT detectors) to visualize antibody binding to CD8 cells in the hPBMC samples. Cells were imaged in the DAPI channel (λ=405), FITC channel (λ=488), Cy3 channel (λ=561) and Cy5 channel (λ=640) using the same image acquisition parameters for each sample (e.g., laser power, master gain, pinhole size, scan speed, offset). All images were analyzed identically using ImageJ.

Antibody efficacy assay. A PD-1/PD-L1 Blockade Bioassay (J1250) was purchased from Promega and was performed according to the commercial protocol. In brief, CHO-K1 artificial antigen presenting cells (aAPC) expressing human PD-L1 and Jurkat T Cells expressing human PD-1 and NFAT-induced luciferase were treated with anti-human-PD-1, anti-human-PD-1-DNA conjugate (i.e., S2-anti-human-PD-1-Cy3-S3), anti-human-PD-1 dimer (i.e., S2-anti-human-PD-1-anti-human-PD-1-S5), and anti-human-PD-1 trimer (i.e., S2-anti-human-PD-1-anti-human-PD-1-anti-human-PD-1-S1). Cells were treated with each sample at antibody concentrations of 167, 66.7, 26.7, 10.7, 4.27, 1.71, 0.683, and 0.273 μM and were incubated at 37° C. for 6 h. The addition of antibodies that block the checkpoint interaction between PD-L1 and PD-1 results in T cell receptor signaling and NFAT-mediated luciferase expression. A luciferase substrate was added to the cell mixture and luciferase activity was quantified by measuring luminescence on a BioTek Cytation 5 plate reader.

Degradation study. An antibody dimer (i.e., S4-anti-mouse-TIGIT-anti-mouse-CTLA-4-S2′) was diluted to antibody concentrations of 125, 50.0, 20.0, 8.00, 3.20, and 1.28 nM in 1×PBS (pH 7.4) buffer containing 10% fetal bovine serum. Samples were incubated at 37° C. for 6 h. After incubation, the degradation of antibody dimers was quantified using analytical agarose gel electrophoresis, where images of agarose gels were analyzed by densitometry using ImageJ to quantify the proportion of dimer and monomer in each sample after incubation.

FIG. 13 shows confocal microscopy images of untreated human peripheral blood mononuclear cells (hPMBCs) with upregulated PD-1 and hPMBCs treated with anti-human Pd1, anti-human PD1-1-DNA conjugate, anti-human-PD1 dimer, and anti-human-PD1 trimer. Cells were imaged for Cy3, Cy5, FITC, and CD*. The Cy3, Cy5, FITC, and CD8 stain images were also merged into one composite image. Images for each sample were taken using the same laser settings and were processed identically.

FIG. 14 show the characterization of antibody function after oligomerization using a PD-1 Blockade Bioassay. The PD-1 blockade bioassay was run with treatments of anti-human-PD-1 (square), anti-human-PD-1-DNA conjugate (circle), anti-human-PD-1 dimer (triangle), and anti-human-PD-1 trimer (star). EC50 values and 95% confidence intervals for EC50 values were calculated for each treatment (see table) and are represented on the plot with dashed lines (lines for anti-human-PD-1 and anti-human-PD-1 trimer are overlaid) and shown in Table 3 below.

TABLE 3
EC₅₀ values and 95% confidence intervals for EC₅₀
values for Antibody Function After Oligomerization

| | | 95% Confidence Interval |
|---|---|---|
| Sample | EC₅₀ (μg/mL) | for EC₅₀ (μg/mL) |
| anti-human-PD-1 | 1.04 | 0.866-1.21 |
| anti-human-PD-1-DNA | 0.868 | 0.761-0.991 |
| conjugate | | |
| anti-human-PD-1 dimer | 0.683 | 0.591-0.789 |
| anti-human-PD-1 trimer | 1.04 | 0.868-1.24 |

FIG. 15 shows the characterization of oligomer degradation in 10% fetal bovine serum. FIGS. 15A and 15B shows the agarose gel characterization of mouse antibody-DNA conjugates, a mouse antibody dimer, and the mouse antibody dimer incubated in 10% FBS at 37° C. for 6 hours at antibody concentrations of 124, 50, 20, 8, 3.2, and 1.28 nM (lanes 4-9, respectively). A single gel was imaged for Cy5 and FITC fluorescence and analyzed by densitometry. The proportion of dimer in the sample decreased from 85% before incubation to 70% after incubation for each sample concentration. FIG. 15C is an identification of the samples.

### REFERENCES

1. Rüker, F.; Wozniak-Knopp, G., Introduction to Antibody Engineering; Springer, 2021.
2. Lu, L. L.; Suscovich, T. J.; Fortune, S. M.; Alter, G. Beyond Binding: Antibody Effector Functions in Infectious Diseases. Nat. Rev. Immunol 2018, 18, 46-61.
3. Chau, C. H.; Steeg, P. S.; Figg, W. D. Antibody-Drug Conjugates for Cancer. The Lancet 2019, 394, 793-804.
4. Hoos, A. Development of Immuno-Oncology Drugs—from CTLA4 to PD1 to the Next Generations. Nat. Rev. Drug Discov. 2016, 15, 235-247.
5. Andrews, L. P.; Yano, H.; Vignali, D. A. A. Inhibitory Receptors and Ligands Beyond PD-1, PD-L1 and CTLA-4: Breakthroughs or Backups. Nat. Immunol. 2019, 20, 1425-1434.
6. Berraondo, P.; Sanmamed, M. F.; Ochoa, M. C.; Etxeberria, I.; Aznar, M. A.; Pérez-Gracia, J. L.; Rodríguez-Ruiz, M. E.; Ponz-Sarvise, M.; Castañón, E.; Melero, I. Cytokines in Clinical Cancer Immunotherapy. Br. J. Cancer 2019, 120, 6-15.
7. Guntern, P.; Eggel, A. Past, Present, and Future of Anti-IgE Biologics. Allergy 2020, 75, 2491-2502.
8. Rotte, A. Combination of CTLA-4 and PD-1 Blockers for Treatment of Cancer. J. Exp. Clin. Cancer Res. 2019, 38, 255.
9. Rodriguez-Abreu, D.; Johnson, M. L.; Hussein, M. A.; Cobo, M.; Patel, A. J.; Secen, N. M.; Lee, K. H.; Massuti, B.; Hiret, S.; Yang, J. C.-H.; Barlesi, F.; Lee, D. H.; Paz-Ares, L. G.; Hsieh, R. W.; Miller, K.; Patil, N.; Twomey, P.; Kapp, A. V.; Meng, R.; Cho, B. C. Primary Analysis of a Randomized, Double-Blind, Phase II Study of the Anti-TIGIT Antibody Tiragolumab (Tira) Plus Atezolizumab (Atezo) Versus Placebo Plus Atezo as First-Line (1L) Treatment in Patients with PD-L1-Selected NSCLC (CITYSCAPE). J. Clin. Oncol. 2020, 38, 9503-9503.
10. Wei, S. C.; Anang, N.-A. A. S.; Sharma, R.; Andrews, M. C.; Reuben, A.; Levine, J. H.; Cogdill, A. P.; Mancuso, J. J.; Wargo, J. A.; Pe'er, D.; Allison, J. P. Combination Anti-CTLA-4 Plus Anti-PD-1 Checkpoint Blockade Utilizes Cellular Mechanisms Partially Distinct from Monotherapies. Proc. Natl. Acad. Sci. U.S.A. 2019, 116, 22699.
11. Nunez-Prado, N.; Compte, M.; Harwood, S.; Alvarez-Mendez, A.; Lykkemark, S.; Sanz, L.; Álvarez-Vallina, L. The Coming of Age of Engineered Multivalent Antibodies. Drug Discov. 2015, 20, 588-594.
12. Modica, J. A.; lderzorig, T.; Mrksich, M. Design and Synthesis of Megamolecule Mimics of a Therapeutic Antibody. J. Am. Chem. Soc. 2020, 142, 13657-13661.
13. Divine, R.; Dang, H. V.; Ueda, G.; Fallas, J. A.; Vulovic, I.; Sheffler, W.; Saini, S.; Zhao, Y. T.; Raj, I. X.; Morawski, P. A.; Jennewein, M. F.; Homad, L. J.; Wan, Y.-H.; Tooley, M. R.; Seeger, F.; Etemadi, A.; Fahning, M. L.; Lazarovits, J.; Roederer, A.; Walls, A. C.; Stewart, L.; Mazloomi, M.; King, N. P.; Campbell, D. J.; McGuire, A. T.; Stamatatos, L.; Ruohola-Baker, H.; Mathieu, J.; Veesler, D.; Baker, D. Designed Proteins Assemble Antibodies into Modular Nanocages. Science 2021, 372, eabd9994.
14. Mack, M.; Riethmüller, G.; Kufer, P. A Small Bispecific Antibody Construct Expressed as a Functional Single-Chain Molecule with High Tumor Cell Cytotoxicity. Proc. Natl. Acad. Sci. U.S.A. 1995, 92, 7021.
15. Moore, P. A.; Zhang, W.; Rainey, G. J.; Burke, S.; Li, H.; Huang, L.; Gorlatov, S.; Veri, M. C.; Aggarwal, S.; Yang, Y.; Shah, K.; Jin, L.; Zhang, S.; He, L.; Zhang, T.; Ciccarone, V.; Koenig, S.; Bonvini, E.; Johnson, S. Application of Dual Affinity Retargeting Molecules to Achieve Optimal Redirected T-cell Killing of B-cell Lymphoma. Blood 2011, 117, 4542-4551.
16. Veggiani, G.; Nakamura, T.; Brenner, M. D.; Gayet, R. V.; Yan, J.; Robinson, C. V.; Howarth, M. Programmable Polyproteams Built Using Twin Peptide Superglues. Proc. Natl. Acad. Sci. U.S.A. 2016, 113, 1202-1207.
17. Wagner, K.; Kwakkenbos, M. J.; Claassen, Y. B.; Maijoor, K.; Bohne, M.; van der Sluijs, K. F.; Witte, M. D.; van Zoelen, D. J.; Cornelissen, L. A.; Beaumont, T.; Bakker, A. Q.; Ploegh, H. L.; Spits, H. Bispecific Antibody Generated with Sortase and Click Chemistry Has Broad Antiinfluenza Virus Activity. Proc. Natl. Acad. Sci. U.S.A. 2014, 111, 16820.
18. Castoldi, R.; Jucknischke, U.; Pradel, L. P.; Arnold, E.; Klein, C.; Scheiblich, S.; Niederfellner, G.; Sustmann, C. Molecular Characterization of Novel Trispecific ErbB-cMet-IGF1R Antibodies and their Antigen-Binding Properties. Protein Engineering, Design and Selection 2012, 25, 551-560.
19. Wu, X.; Demarest, S. J. Building Blocks for Bispecific and Trispecific Antibodies. Methods 2019, 154, 3-9.
20. Kontermann, R. E. Dual Targeting Strategies with Bispecific Antibodies. MAbs 2012, 4, 182-197.
21. Bargou, R.; Leo, E.; Zugmaier, G.; Klinger, M.; Goebeler, M.; Knop, S.; Noppeney, R.; Viardot, A.; Hess, G.; Schuler, M.; Einsele, H.; Brandl, C.; Wolf, A.; Kirchinger, P.; Klappers, P.; Schmidt, M.; Riethmuller, G.; Reinhardt, C.; Baeuerle, P. A.; Kufer, P. Tumor Regression in Cancer Patients by Very Low Doses of a T Cell-Engaging Antibody. Science 2008, 321, 974.
22. Khalili, H.; Godwin, A.; Choi, J.-w.; Lever, R.; Khaw, P. T.; Brocchini, S. Fab-PEG-Fab as a Potential Antibody Mimetic. Bioconj. Chem. 2013, 24, 1870-1882.
23. Hull, E. A.; Livanos, M.; Miranda, E.; Smith, M. E. B.; Chester, K. A.; Baker, J. R. Homogeneous Bispecifics by Disulfide Bridging. Bioconj. Chem. 2014, 25, 1395-1401.
24. Patterson, J. T.; Isaacson, J.; Kerwin, L.; Atassi, G.; Duggal, R.; Bresson, D.; Zhu, T.; Zhou, H.; Fu, Y.; Kaufmann, G. F. PSMA-Targeted Bispecific Fab Conjugates that Engage T Cells. Bioorganic & Medicinal Chemistry Letters 2017, 27, 5490-5495.
25. Maruani, A.; Szijj, P. A.; Bahou, C.; Nogueira, J. C. F.; Caddick, S.; Baker, J. R.; Chudasama, V. A Plug-and-Play Approach for the De Novo Generation of Dually Functionalized Bispecifics. Bioconj. Chem. 2020, 31, 520-529.
26. Scheer, J. M.; Sandoval, W.; Elliott, J. M.; Shao, L.; Luis, E.; Lewin-Koh, S.-C.; Schaefer, G.; Vandlen, R. Reorienting the Fab Domains of Trastuzumab Results in Potent HER2 Activators. PLoS One 2012, 7, e51817.
27. Agarwal, P.; Bertozzi, C. R. Site-Specific Antibody-Drug Conjugates: The Nexus of Bioorthogonal Chemistry, Protein Engineering, and Drug Development. Bioconj. Chem. 2015, 26, 176-192.
28. Szijj, P.; Chudasama, V. The Renaissance of Chemically Generated Bispecific Antibodies. Nat. Rev. Chem. 2021, 5, 78-92.
29. Mirkin, C. A.; Letsinger, R. L.; Mucic, R. C.; Storhoff, J. J. A DNA-Based Method for Rationally Assembling Nanoparticles into Macroscopic Materials. Nature 1996, 382, 607-609.
30. Laramy, C. R.; O'Brien, M. N.; Mirkin, C. A. Crystal Engineering with DNA. Nat. Rev. Mater. 2019, 4, 201-224.
31. Seeman, N. C.; Sleiman, H. F. DNA Nanotechnology. Nat. Rev. Mater. 2017, 3, 17068.
32. Setyawati, M. I.; Kutty, R. V.; Leong, D. T. DNA Nanostructures Carrying Stoichiometrically Definable Antibodies. Small 2016, 12, 5601-5611.
33. Kazane, S. A.; Axup, J. Y.; Kim, C. H.; Ciobanu, M.; Wold, E. D.; Barluenga, S.; Hutchins, B. A.; Schultz, P. G.; Winssinger, N.; Smider, V. V. Self-Assembled Antibody Multimers through Peptide Nucleic Acid Conjugation. J. Am. Chem. Soc. 2013, 135, 340-346.
34. Pan, L.; Cao, C.; Run, C.; Zhou, L.; Chou, J. J. DNA-Mediated Assembly of Multispecific Antibodies for T Cell Engaging and Tumor Killing. Adv. Sci. 2020, 7, 1900973.
35. Coyle, M. P.; Xu, Q.; Chiang, S.; Francis, M. B.; Groves, J. T. DNA-Mediated Assembly of Protein Heterodimers on Membrane Surfaces. J. Am. Chem. Soc. 2013, 135, 5012-5016.
36. Liang, S. I.; McFarland, J. M.; Rabuka, D.; Gartner, Z. J. A Modular Approach for Assembling Aldehyde-Tagged Proteins on DNA Scaffolds. J. Am. Chem. Soc. 2014, 136, 10850-10853.
37. Douglas, S. M.; Bachelet, I.; Church, G. M. A Logic-Gated Nanorobot for Targeted Transport of Molecular Payloads. Science 2012, 335, 831-834.
38. Williams, B. A. R.; Lund, K.; Liu, Y; Yan, H.; Chaput, J. C. Self-Assembled Peptide Nanoarrays: An Approach to Studying Protein-Protein Interactions. Angew. Chem. Int. Ed. 2007, 46, 3051-3054.
39. McMillan, J. R.; Hayes, 0. G.; Winegar, P. H.; Mirkin, C. A. Protein Materials Engineering with DNA. Acc. Chem. Res. 2019, 52, 1939-1948.
40. Stephanopoulos, N. Hybrid Nanostructures from the Self-Assembly of Proteins and DNA. Chem 2020, 6, 364-405.
41. Winegar, P. H.; Hayes, O. G.; McMillan, J. R.; Figg, C. A.; Focia, P. J.; Mirkin, C. A. DNA-Directed Protein Packing within Single Crystals. Chem 2020, 6, 1007-1017.
42. Partridge, B. E.; Winegar, P. H.; Han, Z.; Mirkin, C. A. Redefining Protein Interfaces within Protein Single Crystals with DNA. J. Am. Chem. Soc. 2021, 143, 8925-8934.
43. Subramanian, R. H.; Smith, S. J.; Alberstein, R. G.; Bailey, J. B.; Zhang, L.; Cardone, G.; Suominen, L.; Chami, M.; Stahlberg, H.; Baker, T. S.; Tezcan, F. A. Self-Assembly of a Designed Nucleoprotein Architecture through Multimodal Interactions. ACS Cent. Sci. 2018, 4, 1578-1586.
44. Brodin, J. D.; Auyeung, E.; Mirkin, C. A. DNA-Mediated Engineering of Multicomponent Enzyme Crystals. Proc. Natl. Acad. Sci. U.S.A. 2015, 112, 4564-4569.
45. McMillan, J. R.; Brodin, J. D.; Millan, J. A.; Lee, B.; Olvera de la Cruz, M.; Mirkin, C. A. Modulating Nanoparticle Superlattice Structure Using Proteins with Tunable Bond Distributions. J. Am. Chem. Soc. 2017, 139, 1754-1757.
46. Wang, S.-T.; Minevich, B.; Liu, J.; Zhang, H.; Nykypanchuk, D.; Byrnes, J.; Liu, W.; Bershadsky, L.; Liu, Q.; Wang, T.; Ren, G.; Gang, O. Designed and Biologically Active Protein Lattices. Nat. Commun. 2021, 12, 3702.
47. McMillan, J. R.; Hayes, 0. G.; Remis, J. P.; Mirkin, C. A. Programming Protein Polymerization with DNA. J. Am. Chem. Soc. 2018, 140, 15950-15956.
48. McMillan, J. R.; Mirkin, C. A. DNA-Functionalized, Bivalent Proteins. J. Am. Chem. Soc. 2018, 140, 6776-6779.
49. Kashiwagi, D.; Sim, S.; Niwa, T.; Taguchi, H.; Aida, T. Protein Nanotube Selectively Cleavable with DNA: Supramolecular Polymerization of “DNA-Appended Molecular Chaperones”. J. Am. Chem. Soc. 2018, 140, 26-29.
50. Figg, C. A.; Winegar, P. H.; Hayes, 0. G.; Mirkin, C. A. Controlling the DNA Hybridization Chain Reaction. J. Am. Chem. Soc. 2020, 142, 8596-8601.
51. Mou, Y.; Yu, J.-Y.; Wannier, T. M.; Guo, C.-L.; Mayo, S. L. Computational Design of Co-Assembling Protein-DNA Nanowires. Nature 2015, 525, 230-233.
52. Cheglakov, Z.; Weizmann, Y.; Braunschweig, A. B.; Wilner, 0. I.; Willner, I. Increasing the Complexity of Periodic Protein Nanostructures by the Rolling-Circle-Amplified Synthesis of Aptamers. Angew. Chem. Int. Ed. 2008, 47, 126-130.
53. Meyer, R.; Niemeyer, C. M. Orthogonal Protein Decoration of DNA Nanostructures. Small 2011, 7, 3211-3218.
54. Mogilevsky, C. S.; Lobba, M. J.; Brauer, D. D.; Marmelstein, A. M.; Maza, J. C.; Gleason, J. M.; Doudna, J. A.; Francis, M. B. Synthesis of Multi-Protein Complexes through Charge-Directed Sequential Activation of Tyrosine Residues. J. Am. Chem. Soc. 2021.
55. Hayes, 0. G.; McMillan, J. R.; Lee, B.; Mirkin, C. A. DNA-Encoded Protein Janus Nanoparticles. J. Am. Chem. Soc. 2018, 140, 9269-9274.
56. Sacca, B.; Niemeyer, C. M. Functionalization of DNA Nanostructures with Proteins. Chem. Soc. Rev. 2011, 40, 5910-5921.
57. Chhabra, R.; Sharma, J.; Ke, Y.; Liu, Y.; Rinker, S.; Lindsay, S.; Yan, H. Spatially Addressable Multiprotein Nanoarrays Templated by Aptamer-Tagged DNA Nanoarchitectures. J. Am. Chem. Soc. 2007, 129, 10304-10305.
58. Hamann, P. R.; Hinman, L. M.; Hollander, I.; Beyer, C. F.; Lindh, D.; Holcomb, R.; Hallett, W.; Tsou, H.-R.; Upeslacis, J.; Shochat, D.; Mountain, A.; Flowers, D. A.; Bernstein, I. Gemtuzumab Ozogamicin, A Potent and Selective Anti-CD33 Antibody-Calicheamicin Conjugate for Treatment of Acute Myeloid Leukemia. Bioconj. Chem. 2002, 13, 47-58.
1. Integrated DNA Technologies. OligoAnalyzer. https://www.idtdna.com/pages/tools/oligoanalyzer (accessed Apr. 18, 2021).
2. Hamann, P. R.; Hinman, L. M.; Hollander, I.; Beyer, C. F.; Lindh, D.; Holcomb, R.; Hallett, W.; Tsou, H.-R.; Upeslacis, J.; Shochat, D.; Mountain, A.; Flowers, D. A.; Bernstein, I. Gemtuzumab Ozogamicin, A Potent and Selective Anti-CD33 Antibody-Calicheamicin Conjugate for Treatment of Acute Myeloid Leukemia. Bioconj. Chem. 2002, 13, 47-58.
3. Ikebuchi, R.; Konnai, S.; Okagawa, T.; Yokoyama, K.; Nakajima, C.; Suzuki, Y.; Murata, S.; Ohashi, K. Blockade of Bovine PD-1 Increases T Cell Function and Inhibits Bovine Leukemia Virus Expression in B Cells In Vitro. Vet. Res. 2013, 44, 59.
4. Agata, Y.; Kawasaki, A.; Nishimura, H.; Ishida, Y.; Tsubat, T.; Yagita, H.; Honjo, T. Expression of the PD-1 Antigen on the Surface of Stimulated Mouse T and B Lymphocytes. Int. Immunol. 1996, 8, 765-772.

1. A method of forming a sequence encoded oligomer, comprising:
admixing a first antibody-oligonucleotide conjugate, a second antibody-oligonucleotide conjugate, and at least one template oligonucleotide strand under conditions to hybridize the at least one template oligonucleotide strand to the first and second antibody-oligonucleotide conjugates thereby assembling the first and second oligonucleotide-conjugates into the oligomer, wherein:
the first antibody-oligonucleotide conjugate comprises an oligonucleotide strand comprising a first sequence, a second sequence, and an alkyne,
the second antibody-oligonucleotide conjugate comprises an oligonucleotide strand comprising a third sequence, a fourth sequence, and an alkyne, and
the at least one template oligonucleotide strand comprises at least two template sequences, one of the at least two template sequences being complementary to one of the first and second sequences of the first antibody-oligonucleotide conjugate and the other of the at least two template sequences being complementary to one of the third and fourth sequences of the second antibody-oligonucleotide conjugate, such that the at least one template oligonucleotide strand hybridizes to a portion of the oligonucleotide strand of the first-antibody-oligonucleotide conjugate and to a portion of the oligonucleotide strand of the second antibody.

2. The method of claim 1, wherein the first antibody-oligonucleotide conjugate is formed by reacting a first antibody with an oligo molecule comprising an azide to form a first antibody-azide and reacting the first antibody-azide with the oligonucleotide strand comprising the alkyne to from the first antibody-oligonucleotide conjugate through azide-alkyne cycloaddition reaction; and/or
wherein the second antibody-oligonucleotide conjugate is formed by reacting a second antibody with an oligo molecule comprising an azide to form a second antibody-azide and reacting the second antibody-azide with the oligonucleotide strand comprising the alkyne to from the second antibody-oligonucleotide conjugate through azide-alkyne cycloaddition reaction.

3. (canceled)

4. The method of claim 2, wherein the oligo molecule is one or more of NHS-PEG₁₂-N₃, NHS-PEG₄-azide, NHS-azide, N-5-azido-2-nitrobenzoyloxysuccinimide, and sulfosuccinimidyl-6-[4′-azido-2′-nitrophenylamino]hexanoate.

5. The method of claim 1, wherein any one or more of the first, second, third, and fourth sequences comprises at least 20 bases.

6. The method of claim 1, wherein the first antibody-oligonucleotide conjugate further comprises a dye attached to the oligonucleotide and/or wherein the second antibody-oligonucleotide conjugate further comprises a dye attached to the oligonucleotide.

7. (canceled)

8. The method of claim 1, further comprising admixing a third antibody-oligonucleotide conjugate and at least two template oligonucleotide strands with the first and second antibody-oligonucleotide conjugates, wherein the third antibody-oligonucleotide conjugate comprises an oligonucleotide strand with a fifth sequence joined to a sixth sequence by an alkyne, the at least two template oligonucleotide strands each comprise at least two template sequences complementary to one of the first to sixth sequences such that each of the at least two template oligonucleotide strands hybridizes to portions of oligonucleotide strands of two different ones of the first, second, and third antibody-oligonucleotide conjugates.

9. The method of claim 1, wherein the alkyne is dibenzocyclooctyne.

10. The method of claim 1, wherein each of the at least one template oligonucleotide strands comprises a toehold sequence, optionally wherein the toehold sequence is 8 bases.

11. (canceled)

12. The method of claim 1, wherein in the oligonucleotide strand of the first antibody-oligonucleotide conjugate, the alkyne is arranged between the first and second sequence and/or wherein in the oligonucleotide strand of the second antibody-oligonucleotide conjugate, the alkyne is arranged between the third and fourth sequence.

13. (canceled)

14. A method of forming a sequence-encoded oligomer, comprising:
admixing at least two antibody-oligonucleotide conjugates with at least one template oligonucleotide strand under conditions to hybridize portions of the at least one template oligonucleotide strand with a hybridizing portion on each of the at least two antibody-oligonucleotide conjugates to form the oligomer, wherein:
each of the at least two antibody-oligonucleotide conjugates comprises an antibody attached to an oligonucleotide strand comprising an alkyne and the hybridizing portion, and
each of the at least one template oligonucleotide comprises two complementary sequence regions, each complementary sequence region having a sequence complementary to the hybridizing portion of different ones of the at least two antibody-oligonucleotide conjugates.

15. The method of claim 14, wherein the alkyne is dibenzocyclooctyne.

16. The method of claim 14 or 15, wherein the oligonucleotide strand of each of the at least two antibody-oligonucleotide conjugates comprises a dye.

17. The method of claims 14 to 16, wherein the antibody is attached to the oligonucleotide strand through an azide-alkyne reaction.

18. The method of claim 17, wherein the antibody is modified with one or more of NHS-PEG₁₂-N₃, NHS-PEG₄-azide, NHS-azide, N-5-azido-2-nitrobenzoyloxysuccinimide, and sulfosuccinimidyl-6-[4′-azido-2′-nitrophenylamino]hexanoate prior to attachment of the oligonucleotide strand.

19. The method of claim 14, comprising admixing at least three antibody-oligonucleotide conjugates and at least two template oligonucleotide strands.

20. A method of forming a sequence-encoded oligomer, comprising:
admixing at least two antibody-oligonucleotide conjugates with at least one template oligonucleotide strand under conditions to hybridize portions of the at least one template oligonucleotide strand with a hybridizing portion on each of the at least two antibody-oligonucleotide conjugates to form a first oligomer;
admixing at least two antibody-oligonucleotide conjugates with at least one template oligonucleotide strand under conditions to hybridize portions of the at least one template oligonucleotide strand with a hybridizing portion on each of the at least two antibody-oligonucleotide conjugates to form a second oligomer, wherein each of the first and second oligomer comprises an oligonucleotide strand having an oligomer hybridizing portion; and
admixing the first and second oligomers with at least one oligomer template oligonucleotide strand under conditions to hybridize portions of the at least one oligomer template oligonucleotide strand with the oligomer hybridizing portions of the first and second oligomers to form a third oligomer,
wherein:
the at least one oligomer template oligonucleotide strand comprises two complementary sequence regions, each complementary sequence region having a sequence complementary to a respective one of the oligomer hybridizing portion of the first and second oligomers,
each of the at least two antibody-oligonucleotide conjugates comprises an antibody attached to an oligonucleotide strand comprising an alkyne and the hybridizing portion, and
each of the at least one template oligonucleotide comprises two complementary sequence regions, each complementary sequence region having a sequence complementary to the hybridizing portion of different ones of the at least two antibody-oligonucleotide conjugates.

21. The method of claim 20, wherein the alkyne is dibenzocyclooctyne.

22. (canceled)

23. The method of claims 20 to 22, wherein the antibody is attached to the oligonucleotide strand through an azide-alkyne reaction.

24. The method of claim 23, wherein the antibody is modified with one or more of NHS-PEG₁₂-N₃, NHS-PEG₄-azide, NHS-azide, N-5-azido-2-nitrobenzoyloxysuccinimide, and sulfosuccinimidyl-6-[4′-azido-2′-nitrophenylamino]hexanoate prior to attachment of the oligonucleotide strand.

25. The method of claim 1, wherein in any of the antibody-oligonucleotide conjugates or template oligonucleotides, the oligonucleotide is DNA or RNA.
